(19) United States
(12) Patent Application Publication
(10) Pub. No.: US 2012/0046735 A1
(43) Pub. Date: Feb. 23, 2012

(54) PLASMA MODIFICATION OF METAL SURFACES

(75) Inventors: Kevin N. Sill, Tampa, FL (US); Habib Skaff, Tampa, FL (US); Kurt Breitenkamp, San Diego, CA (US)

(73) Assignee: Intezyne Technologies, Incorporated, Tampa, FL (US)

(21) Appl. No.: 13/007,455

(22) Filed: Jan. 14, 2011

Related U.S. Application Data
(60) Provisional application No. 61/295,367, filed on Jan. 15, 2010.

(51) Int. Cl. A61F 2/06; B32B 15/04; C08G 65/333; B05D 3/10; B05D 7/24

(52) U.S. Cl. 623/1.46; 427/302; 427/299; 525/409; 428/457

(57) ABSTRACT

The present invention provides modified metal surfaces, methods of preparing the same, and intermediates thereto. These materials are useful in a variety of applications including biomaterials.

### CROSS-REFERENCE TO RELATED APPLICATIONS

The present application claims priority to U.S. provisional patent application U.S. Ser. No. 61/295,367, filed Jan. 15, 2010, the entirety of which is incorporated herein by reference.

### BACKGROUND OF THE INVENTION

Modification of inorganic substrates with polymeric materials has been utilized in a range of applications across numerous scientific disciplines including analytical chemistry, biology, and electronics. (Mansky, P., et. al. Science 1997, 275, 1458-1460; Huang, Z. Langmuir, 1997, 13, 6480-6484. Granick, S. et. al. J. Polym. Sci. B. 2003, 41, 2755-2793.) Inorganic substrates can be coated with polymers or other molecules using a number of currently available methods. One popular, simple method involves the physical adsorbtion of a polymer to a substrate through coating or other deposition techniques. Other methods utilize covalent or ionic bonding between functionality on a polymer, or small molecule, and functionality present on the substrate surface to achieve modification. (Denes, A. R. et. al. J. Appl. Polym. Sci. 2001, 81, 3425-3438). While simple adsorption of polymers to metal substrates has proven successful in many cases, this procedure does not produce mechanically robust coatings with long-term stability. Post-adsorption crosslinking (Dong, B. et. al.; J. Appl. Polym. Sci., 2005, 97, 485-497.) of the polymer coating may increase the toughness and short-term performance of the resulting film, but such crosslinking can also result in cracking and flaking of the polymer films over time, resulting in mechanical failure and a dramatic reduction in film properties. The chemical attachment of functional polymers to a metal substrate introduces a stable, robust linkage between polymer chains and the metal substrate and represents a more desirable scenario for many applications where the long-term stability of the coating is required for optimal performance. (Hara, H. et. al. Adv. Drug Del. Rev. 2006, 58, 377-388.) However, the methodologies to prepare covalent attachment of polymers to metallic and non-metallic substrates has thus far been limited to only a few examples of suitable substrates and complimentary chemical functionalities. Such examples include the near-covalent interaction between gold substrates and thiol-functionalized molecules, covalent bonds formed between silica and alcohol, silyl chloride, or silyl alcohol-functionalized compounds, and covalent bonds formed between hydrogen-functionalized silicon surfaces and alkene-substituted molecules. (Mansky, P., et. al. Science 1997, 275, 1458-1460, Pesek, J. J.; Matyska, M. T. Interface Science 1997, 5, 103-117.)

Accordingly, it would be advantageous to provide a method of modifying metal surfaces to provide a metal substrate capable of forming covalent bonds with appropriately functionalized polymers or small molecule derivatives.

### BRIEF DESCRIPTION OF THE DRAWINGS

FIG. 1 depicts methods of incorporating amine groups onto a metal substrate.

FIG. 2 depicts a method for covalently modifying amine groups incorporated onto a metal surface via condensation reaction with an acyl halide.

FIG. 3 depicts a method for covalently modifying amine groups incorporated onto a metal surface via condensation reaction with an isocyanate.

FIG. 4 depicts a method for covalently modifying amine groups incorporated onto a metal surface via alkylation with an alkyl halide.

FIG. 5 depicts a method for covalently modifying amine groups incorporated onto a metal surface via acylation with an anhydride.

FIG. 6 depicts a method for covalently modifying amine groups incorporated onto a metal surface via Michael addition.

FIG. 7 depicts a method for covalently modifying amine groups incorporated onto a metal surface via condensation with an aldehyde.

FIG. 8 depicts a method for covalently modifying thiol groups incorporated onto a metal surface with thiols to produce disulfides.

FIG. 9 depicts a method for covalently modifying thiol groups incorporated onto a metal surface via Michael addition.

FIG. 10 depicts a method for covalently modifying amine groups incorporated onto a metal surface via condensation with a isocyanate-terminated polymer.

FIG. 11 depicts a method for covalently modifying amine groups incorporated onto a metal surface via condensation with an acyl halide-terminated polymer.

FIG. 12 depicts a method for covalently modifying amine groups incorporated onto a metal surface via condensation.

### DETAILED DESCRIPTION OF CERTAIN EMBODIMENTS OF THE INVENTION

### 1. General Description of the Invention

The present invention provides methods for covalently modifying a metal surface with a polymeric group or a small molecule organic moiety. In order to covalently bond the polymeric group or small molecule organic moiety to the metal surface, the metal surface is treated to introduce amine or thiol groups. In certain embodiments, the present invention provides a method for covalently modifying a metal surface, comprising the steps of introducing amine or thiol groups onto a metal substrate and covalently bonding a polymer or small molecule organic moiety onto the resulting hydrophilic metal surface. In some embodiments, the metal surface is treated to introduce amine groups. In other embodiments, the metal surface is treated to introduce thiol groups.

### 2. Definitions

Compounds of this invention include those described generally above, and are further illustrated by the embodiments, sub-embodiments, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

As used herein, the term “sequential polymerization”, and variations thereof, refers to the method where after a first monomer (e.g. NCA or lactam) is incorporated into the polymer, thus forming a “block”, a second monomer (e.g. NCA or lactam) is added to the reaction and the polymerization continues in a similar fashion resulting in the formation of multi-block copolymers.

As used herein, the term “block copolymer” refers to a polymer comprising two or more polymer portions. The term “multi-block copolymer” refers to a polymer comprising at least three separate polymer portions. These are also referred to as triblock copolymers, tetrablock copolymers, etc. Such multi-block copolymers may be of the format X-T-X′, T-X-X′, T-X-X′-X″ or X′-X-T-X-X′, wherein T is a certain synthetic polymer portion and X, X′, and X″ are differing polymer chains. In certain aspects, the synthetic polymer is used as the center block which allows the growth of multiple blocks symmetrically from center.

As used herein, the term “synthetic polymer” refers to a polymer that is well known in the art and includes polystyrene, polyalkylene oxides, polyacrylates, polyacrylamides, polyamines, polyolefins, and derivatives thereof.

As used herein, the term “natural polymer” refers to a polymer that is well known in the art and includes polysaccarides, dextran, heparin, fibronectin, poly(amino acids), starch, amylose, amylopectin, polypeptides, proteins, and derivatives thereof.

As used herein, the term “polymer” may refer to either a natural polymer or synthetic polymer.

As used herein, the term “poly(amino acid)” or “amino acid block” refers to a covalently linked amino acid chain wherein each monomer is an amino acid unit. Such amino acid units include natural and unnatural amino acids. In certain embodiments, each amino acid unit is in the L-configuration. Such poly(amino acids) include those having suitably protected functional groups. For example, amino acid monomers may have hydroxyl or amino moieties which are optionally protected by a suitable hydroxyl protecting group or a suitable amine protecting group, as appropriate. Such suitable hydroxyl protecting groups and suitable amine protecting groups are described in more detail herein, infra. As used herein, an amino acid block comprises one or more monomers or a set of two or more monomers. In certain embodiments, an amino acid block comprises one or more monomers such that the overall block is hydrophilic. In other embodiments, an amino acid block comprises one or more monomers such that the overall block is hydrophobic. In still other embodiments, amino acid blocks of the present invention include random amino acid blocks, ie blocks comprising a mixture of amino acid residues.

As used herein, the phrase “natural amino acid side-chain group” refers to the side-chain group of any of the 20 amino acids naturally occuring in proteins. Such natural amino acids include the nonpolar, or hydrophobic amino acids, glycine, alanine, valine, leucine isoleucine, methionine, phenylalanine, tryptophan, and proline. Cysteine is sometimes classified as nonpolar or hydrophobic and other times as polar. Natural amino acids also include polar, or hydrophilic amino acids, such as tyrosine, serine, threonine, aspartic acid (also known as aspartate, when charged), glutamic acid (also known as glutamate, when charged), asparagine, and glutamine. Certain polar, or hydrophilic, amino acids have charged side-chains. Such charged amino acids include lysine, arginine, and histidine. One of ordinary skill in the art would recognize that protection of a polar or hydrophilic amino acid side-chain can render that amino acid nonpolar. For example, a suitably protected tyrosine hydroxyl group can render that tyroine nonpolar and hydrophobic by virtue of protecting the hydroxyl group.

As used herein, the phrase “unnatural amino acid side-chain group” refers to amino acids not included in the list of 20 amino acids naturally occuring in proteins, as described above. Such amino acids include the D-isomer of any of the 20 naturally occuring amino acids. Unnatural amino acids also include homoserine, ornithine, and thyroxine. Other unnatural amino acids side-chains are well know to one of ordinary skill in the art and include unnatural aliphatic side chains. Other unnatural amino acids include modified amino acids, including those that are N-alkylated, cyclized, phosphorylated, acetylated, amidated, azidylated, labelled, and the like.

As used herein, the phrase “living polymer chain-end” refers to the terminus resulting from a polymerization reaction having maintained chain-end reactivity after the completion of the reaction.

As used herein, the term “termination” refers to attaching a terminal group to a polymer chain-end by the reaction of a living polymer with an appropriate compound. Alternatively, the term “termination” may refer to attaching a terminal group to a hydroxyl end, or derivative thereof, of the polymer chain.

As used herein, the term “polymerization terminator” is used interchangeably with the term “polymerization terminating agent” and refers to a compound for attaching a terminal group to a polymer chain-end of a living polymer. Alternatively, the term “polymerization terminator” may refer to a compound for attaching a terminal group to a hydroxyl end, or derivative thereof, of the polymer chain.

As used herein, the term “polymerization initiator” refers to a compound, or anion thereof, which reacts with the desired monomer in a manner which results in polymerization of that monomer. In certain embodiments, the polymerization initiator is the anion of a functional group which initiates the polymerization of ethylene oxide. In other embodiments, the polymerization initiator is the amine salt described herein.

As used herein, the term “Michael acceptor” refers to a compound, or moiety, which is the electrophilic reactant in a Michael reaction with an amine or thiol group incorporated onto a metal surface in accordance with the present invention. Michael acceptors are well known in the art and include maleimide and other electrophilic olefins.

The term “targeting group”, as used herein refers to any molecule, macromolecule, or biomacromolecule which selectively binds to receptors that are over-expressed on specific cell types. Such molecules can be attached to the functionalized end-group of a PEG for cell specific delivery of proteins, viruses, DNA plasmids, oligonucleotides (e.g. siRNA, miRNA, antisense therapeutics, aptamers, etc.), drugs, dyes, and primary or secondary labels which are bound to the opposite PEG end-group. Such targeting groups include, but or not limited to monoclonal and polyclonal antibodies (e.g. IgG, IgA, IgM, IgD, IgE antibodies), sugars (e.g. mannose, mannose-6-phosphate, galactose), proteins (e.g. transferrin), oligopeptides (e.g. cyclic and acylic RGD-containing oligopedptides), oligonucleotides (e.g. aptamers), and vitamins (e.g. folate).

As described herein, compounds of the invention may optionally be substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-20 aliphatic carbon atoms. In some embodiments, aliphatic groups contain 1-10 aliphatic carbon atoms. In other embodiments, aliphatic groups contain 1-8 aliphatic carbon atoms. In still other embodiments, aliphatic groups contain 1-6 aliphatic carbon atoms, and in yet other embodiments aliphatic groups contain 1-4 aliphatic carbon atoms. In some embodiments, “cycloaliphatic” (or “carbocycle” or “cycloalkyl”) refers to a monocyclic C₃-C₈ hydrocarbon or bicyclic C₈-C₁₂ hydrocarbon that is completely saturated or that contains one or more units of unsaturation, but which is not aromatic, that has a single point of attachment to the rest of the molecule wherein any individual ring in said bicyclic ring system has 3-7 members. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heterocycle”, “heterocyclyl”, “heterocycloaliphatic”, or “heterocyclic” as used herein means non-aromatic, monocyclic, bicyclic, or tricyclic ring systems in which one or more ring members is an independently selected heteroatom. In some embodiments, the “heterocycle”, “heterocyclyl”, “heterocycloaliphatic”, or “heterocyclic” group has three to fourteen ring members in which one or more ring members is a heteroatom independently selected from oxygen, sulfur, nitrogen, or phosphorus, and each ring in the system contains 3 to 7 ring members.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl).

The term “unsaturated”, as used herein, means that a moiety has one or more units of unsaturation.

The term “alkoxy”, or “thioalkyl”, as used herein, refers to an alkyl group, as previously defined, attached to the principal carbon chain through an oxygen (“alkoxy”) or sulfur (“thioalkyl”) atom.

The terms “haloalkyl”, “haloalkenyl” and “haloalkoxy” means alkyl, alkenyl or alkoxy, as the case may be, substituted with one or more halogen atoms. The term “halogen” means F, Cl, Br, or I.

The term “aryl” used alone or as part of a larger moiety as in “aralkyl”, “aralkoxy”, or “aryloxyalkyl”, refers to monocyclic, bicyclic, and tricyclic ring systems having a total of five to fourteen ring members, wherein at least one ring in the system is aromatic and wherein each ring in the system contains 3 to 7 ring members. The term “aryl” may be used interchangeably with the term “aryl ring”. The term “aryl” also refers to heteroaryl ring systems as defined hereinbelow.

The term “heteroaryl”, used alone or as part of a larger moiety as in “heteroaralkyl” or “heteroarylalkoxy”, refers to monocyclic, bicyclic, and tricyclic ring systems having a total of five to fourteen ring members, wherein at least one ring in the system is aromatic, at least one ring in the system contains one or more heteroatoms, and wherein each ring in the system contains 3 to 7 ring members. The term “heteroaryl” may be used interchangeably with the term “heteroaryl ring” or the term “heteroaromatic”.

An aryl (including aralkyl, aralkoxy, aryloxyalkyl and the like) or heteroaryl (including heteroaralkyl and heteroarylalkoxy and the like) group may contain one or more substituents. Suitable substituents on the unsaturated carbon atom of an aryl or heteroaryl group are selected from halogen; N₃, CN, R°; OR°; SR°; 1,2-methylene-dioxy; 1,2-ethylenedioxy; phenyl (Ph) optionally substituted with R°; —O(Ph) optionally substituted with R°; (CH₂)₁₋₂(Ph), optionally substituted with R°; CH═CH(Ph), optionally substituted with R°; NO₂; CN;)N(R°)₂; NR°C(O)R°; NR°C(O)N(R°)₂; NR°CO₂R°; —NR°NR°C(O)R°; NR°NR°C(O)N(R°)₂; NR°NR°CO₂R°; C(O)C(O)R°; C(O)CH₂C(O)R°; CO₂R°; C(O)R°; C(O)N(R°)₂; OC(O)N(R°)₂; S(O)₂R°; SO₂N(R°)₂; S(O)R°; NR°SO₂N(R°)₂; NR°SO₂R°; C(═S)N(R°)₂; C(═NH)—N(R°)₂; or (CH₂)₀₋₂NHC(O)R° wherein each independent occurrence of R° is selected from hydrogen, optionally substituted C₁₋₆ aliphatic, an unsubstituted 5-6 membered heteroaryl or heterocyclic ring, phenyl, O(Ph), or CH₂(Ph), or, notwithstanding the definition above, two independent occurrences of R°, on the same substituent or different substituents, taken together with the atom(s) to which each R° group is bound, form a 3-8 membered cycloalkyl, heterocyclyl, aryl, or heteroaryl ring having 0-4 heteroatoms independently selected from nitrogen, oxygen, or sulfur. Optional substituents on the aliphatic group of R° are selected from N₃, CN, NH₂, NH(C₁₋₄aliphatic), N(C₁₋₄aliphatic)₂, halogen, C₁₋₄aliphatic, OH, O(C₁₋₄aliphatic), NO₂, CN, CO₂H, CO₂(C₁₋₄aliphatic), O(haloC₁₋₄ aliphatic), or haloC₁₋₄aliphatic, wherein each of the foregoing C₁₋₄aliphatic groups of R° is unsubstituted.

An aliphatic or heteroaliphatic group or a non-aromatic heterocyclic ring may contain one or more substituents. Suitable substituents on the saturated carbon of an aliphatic or heteroaliphatic group, or of a non-aromatic heterocyclic ring are selected from those listed above for the unsaturated carbon of an aryl or heteroaryl group and additionally include the following: ═O, ═S, ═NNHR*, ═NN(R*)₂, ═NNHC(O)R*, ═NNHCO₂(alkyl), ═NNHSO₂(alkyl), or ═NR*, where each R* is independently selected from hydrogen or an optionally substituted C₁₋₆ aliphatic. Optional substituents on the aliphatic group of R* are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R* is unsubstituted.

Optional substituents on the nitrogen of a non-aromatic heterocyclic ring are selected from R⁺, N(R⁺)₂, C(O)R⁺, CO₂R⁻, C(O)C(O)R⁺, C(O)CH₂C(O)R⁺, SO₂R⁺, SO₂N(R⁺)₂, C(═S)N(R⁺)₂, C(═NH)—N(R⁺)₂, or NR⁺SO₂R⁺; wherein R⁺ is hydrogen, an optionally substituted C₁₋₆ aliphatic, optionally substituted phenyl, optionally substituted O(Ph), optionally substituted CH₂(Ph), optionally substituted (CH₂)₁₋₂(Ph); optionally substituted CH═CH(Ph); or an unsubstituted 5-6 membered heteroaryl or heterocyclic ring having one to four heteroatoms independently selected from oxygen, nitrogen, or sulfur, or, notwithstanding the definition above, two independent occurrences of R⁺, on the same substituent or different substituents, taken together with the atom(s) to which each R⁺ group is bound, form a 3-8-membered cycloalkyl, heterocyclyl, aryl, or heteroaryl ring having 0-4 heteroatoms independently selected from nitrogen, oxygen, or sulfur. Optional substituents on the aliphatic group or the phenyl ring of R⁺ are selected from NH₂, NH(C₁₋₄ aliphatic), N(C₁₋₄ aliphatic)₂, halogen, C₁₋₄ aliphatic, OH, O(C₁₋₄ aliphatic), NO₂, CN, CO₂H, CO₂(C₁₋₄ aliphatic), O(halo C₁₋₄ aliphatic), or halo(C₁₋₄ aliphatic), wherein each of the foregoing C₁₋₄aliphatic groups of R⁺ is unsubstituted.

As detailed above, in some embodiments, two independent occurrences of R° (or R⁺, or any other variable similarly defined herein), are taken together together with the atom(s) to which each variable is bound to form a 3-8-membered cycloalkyl, heterocyclyl, aryl, or heteroaryl ring having 0-4 heteroatoms independently selected from nitrogen, oxygen, or sulfur. Exemplary rings that are formed when two independent occurrences of R° (or R⁺, or any other variable similarly defined herein) are taken together with the atom(s) to which each variable is bound include, but are not limited to the following: a) two independent occurrences of R° (or R⁺, or any other variable similarly defined herein) that are bound to the same atom and are taken together with that atom to form a ring, for example, N(R°)₂, where both occurrences of R° are taken together with the nitrogen atom to form a piperidin-1-yl, piperazin-1-yl, or morpholin-4-yl group; and b) two independent occurrences of R° (or R⁺, or any other variable similarly defined herein) that are bound to different atoms and are taken together with both of those atoms to form a ring, for example where a phenyl group is substituted with two occurrences of OR°

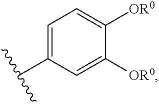

these two occurrences of R° are taken together with the oxygen atoms to which they are bound to form a fused 6-membered oxygen containing ring:

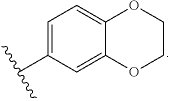

It will be appreciated that a variety of other rings can be formed when two independent occurrences of R° (or R⁺, or any other variable similarly defined herein) are taken together with the atom(s) to which each variable is bound and that the examples detailed above are not intended to be limiting.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, the term “detectable moiety” is used interchangeably with the term “label” and relates to any moiety capable of being detected, e.g., primary labels and secondary labels. Primary labels, such as radioisotopes (e.g., ³²P, ³³P, ³⁵S, or ¹⁴C), mass-tags, and fluorescent labels are signal generating reporter groups which can be detected without further modifications.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The term “metal substrate”, as used herein refers to any metallic material which may be modified to incorporate amine or thiol groups to which a functionalized end-group of a polymeric or small molecule organic group can be attached.

The term “leaving group”, as used herein is a chemical moiety that is readily displaced by a desired incoming chemical moiety. Suitable leaving groups are well known in the art, e.g., see, “Advanced Organic Chemistry,” Jerry March, 4th Ed., pp. 351-357, John Wiley and Sons, N.Y. (1992). Such leaving groups include, but are not limited to, halogen, alkoxy, sulphonyloxy, optionally substituted alkylsulphonyloxy, optionally substituted alkenylsulfonyloxy, optionally substituted arylsulfonyloxy, and diazonium moieties. Examples of suitable leaving groups include chloro, iodo, bromo, fluoro, methanesulfonyl(mesyl), tosyl, triflate, nitro-phenylsulfonyl(nosyl), and bromo-phenylsulfonyl(brosyl). According to an alternate embodiment, the suitable leaving group may be generated in situ within the reaction medium. For example, a leaving group may be generated in situ from a precursor of that compound wherein said precursor contains a group readily replaced by said leaving group in situ.

As used herein, the term “activated ester” refers to an ester moiety which can react with an amine or thiol group incorporated onto the metal surface. Such activated esters are well known in the art and include N-hydroxysuccinimide esters and Mukaiyama esters. In some embodiments, the activated ester is formed by treatment of a carboxylic acid with an activating agent, such as dicyclohexylcarbodiimide (DCC) and diisopropylcarbodiimide (DIC), to form a highly reactive O-acyl-urea. Numerous other activating agents are well known in the art and include HOBt, HOAt, HBTU, HATU, PyBOP and the like.

### 3. Description of Exemplary Embodiments

As described generally above, the present invention provides a method for covalently modifying a metal substrate, comprising the steps of introducing amine or thiol groups onto the metal substrate to produce a hydrophilic metal surface and covalently bonding a polymer or small molecule organic moiety onto the hydrophilic metal surface. As used herein, the phrase “hydrophilic metal surface” refers to a metal substrate onto which a plurality of amine or thiol groups has been incorporated. One of ordinary skill in the art would recognize that various metallic substrates are amenable to methods of the present invention. In certain embodiments, the metal substrate is any such substrate that comprises iron. In other embodiments, the metal substrate comprises a stainless steel, a cobalt alloy, or a titanium alloy. In still other embodiments, the metal substrate comprises iron, iron alloys, steel, stainless steel, austenitic stainless steel, Type 316 stainless steel, ferritic stainless steel, martensitic stainless steel, duplex stainless steel, cobalt, cobalt alloys, cobalt-chromium alloys, stellite alloys, Vitallium®, titanium, titanium alloys, nickel-titanium alloys, nitinol, or super-alloys.

Hydrophilic metal surfaces can be prepared with the use of ammonia plasmas, nitrogen plasmas, or H₂S plasmas. In some embodiments, the plasma is a cold plasma. In certain embodiments, the metal substrate is treated with ammonia cold plasma. Methods of treating a metal substrate with plasma, and cold plasma, are well known in the art. Such methods are described in more detail in the Examples section, infra.

As described generally above, the functionalized metal surface is covalently bonded to a polymer or a small molecule organic moiety. One of ordinary skill in the art would appreciate that amine and thiol groups are covalently bonded to a variety of other functional groups (e.g., with carboxylic acids to form amides and thioesters thereof) by condensation, dehydration, or other coupling reaction. All such functional groups capable of covalently bonding to amine and thiol groups incorporated onto the metal surface are contemplated. In certain embodiments, the polymer or small molecule organic moiety comprises one or more functional groups capable of covalently bonding to one or more amine or thiol groups incorporated onto the metal surface. Exemplary functional groups include, but are not limited to, phosphonic acids, carboxylic acids, anhydrides, isocyanates, Michael acceptors, aldehydes, phosphonic halides, and acyl halides. One of ordinary skill in the art will recognize that thiol functional groups can react with thiol groups incorporated onto the metal surface to afford disulfide bonds.

Polymeric groups for use in the present invention comprise one or more functional groups capable of covalently bonding with one or more amine or thiol groups incorporated onto the metal surface. It will be appreciated that many such polymeric groups are amenable to this reaction. These polymeric groups include natural or synthetic polymers and copolymers. Exemplary polymers include mono-functionalized PEG's, poly(amino acids), heterobifunctional PEG's, branched PEG's, heterofunctionalized branched PEG's, PEG-b-PAA-b-PAA block copolymer, PEG-b-PAA-b-PEG block copolymers, PEG-b-polyester-b-PEG block copolymers, PEG-b-PAA block copolymers, [where PAA refers to poly(amino acid)], dextran, heparin, fibronectin, chitosan, amylose, amylopectin, glycogen, xanthan, gellan, pullulun, cellulose, and cellulose acetate.

In certain embodiments, the present invention provides a method for preparing a covalently modified metal surface, comprising the steps of:
(a) modifying a metal substrate to incorporate thereon a plurality of amine groups;
(b) providing a compound of formula I:

R¹—W I

wherein:
R¹ is a natural or synthetic polymer or copolymer group or a small molecule organic group;
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
   and
(c) coupling the compound of formula I to one or more of the amine groups on the metal surface.

In other embodiments, the present invention provides a method for preparing a covalently modified metal surface, comprising the steps of:
(a) modifying a metal substrate to incorporate thereon a plurality of thiol groups;
(b) providing a compound of formula I:

R¹—W I

wherein:
R¹ is a natural or synthetic polymer or copolymer group or a small molecule organic group;
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
   and
(c) coupling the compound of formula I to one or more of the thiol groups on the metal surface.

As described generally above, the coupling step (c) can be performed in the absence of reagents or catalysts by dehydration or condensation. However, it is also contemplated that the coupling step (c) can be performed in the presence of such reagents or catalysts. For example, coupling step (c) may be performed in the presence of a suitable base. Suitable bases include any of those known to one of ordinary skill in the art for such coupling reactions. Exemplary bases include, but are not limited to, inorganic and organic bases including amine bases,such as triethylamine, diisopropylamine, diisopropylethylamine, dimethylaminopyridine, and the like.

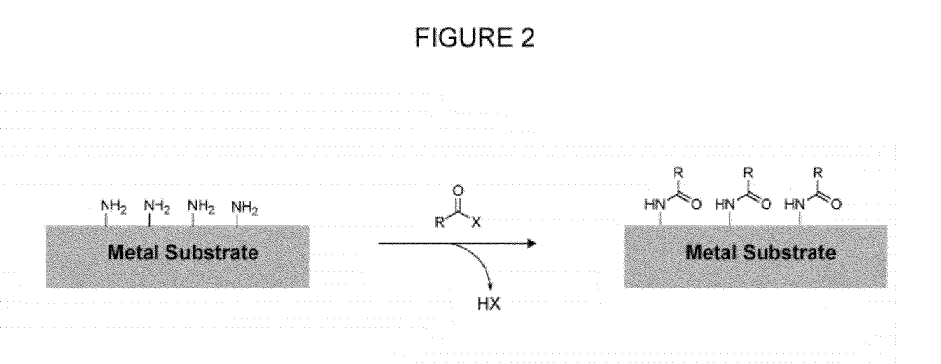

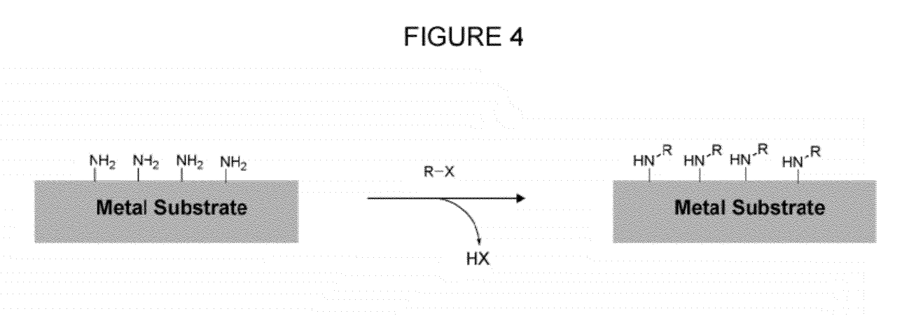

One of ordinary skill in the art will appreciate that when W is —C(═O)X, —P(═O)(X)₂, —P(═O)(OR^{a})X, —P(═O)(R^{a})X, then the coupling at step (c) can occur by a condensation reaction. See FIGS. 2, 4, 12 which depict representative methods of the present invention whereby coupling step (c) occurs by condensation reaction.

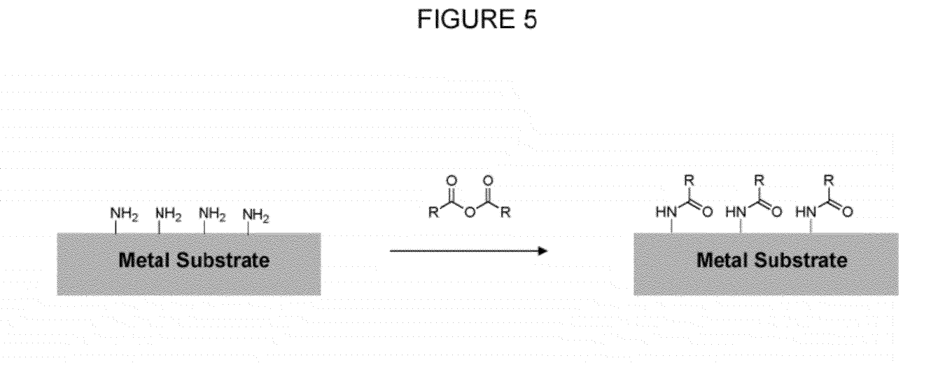

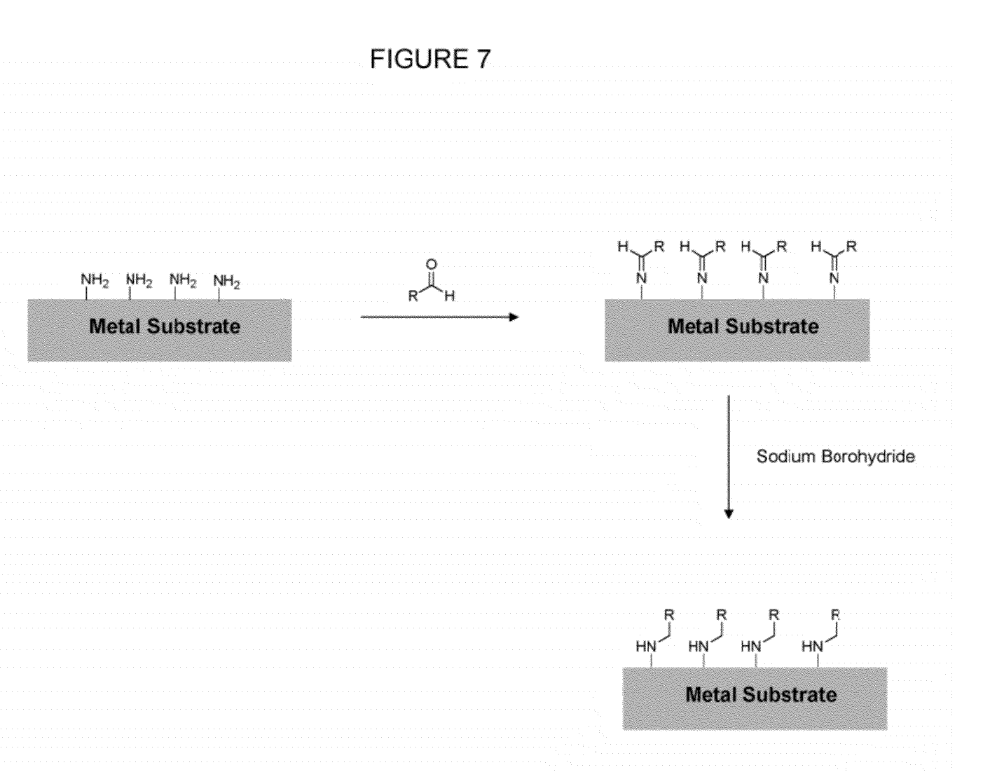

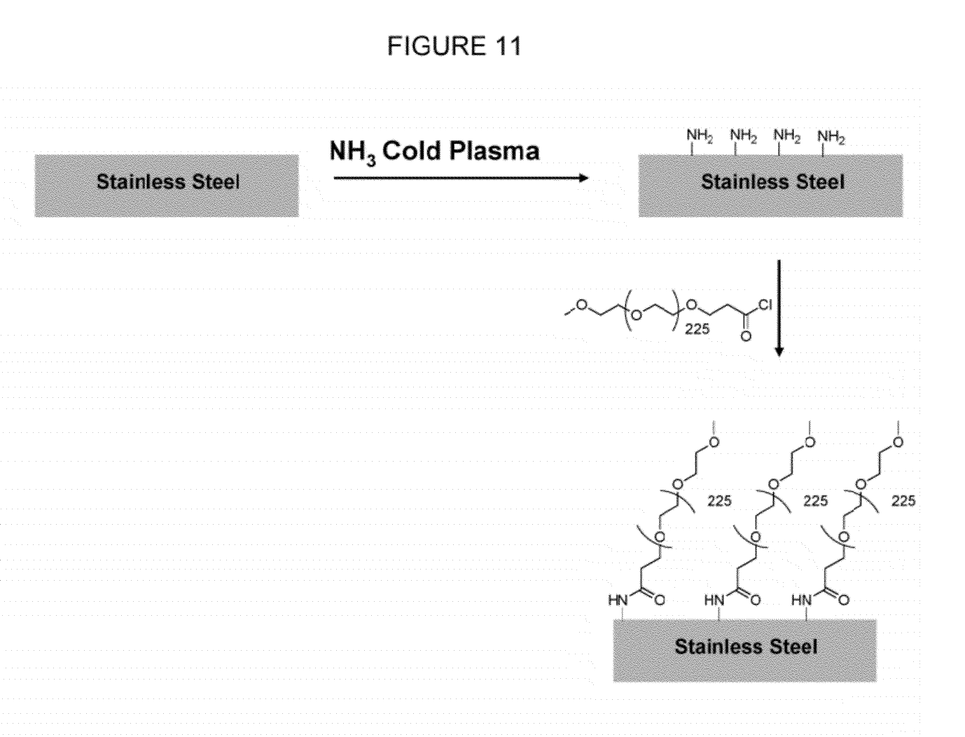

Similarly, when W is —C(═O)OH, —P(═O)(OH)₂, —P(═O)(R^{a})OH, or —P(═O)(OR^{a})OH the coupling at step (c) can occur by a dehydration reaction. See FIGS. 5, 7, and 11 which depict representative methods of the present invention whereby coupling step (c) occurs by dehydration reaction.

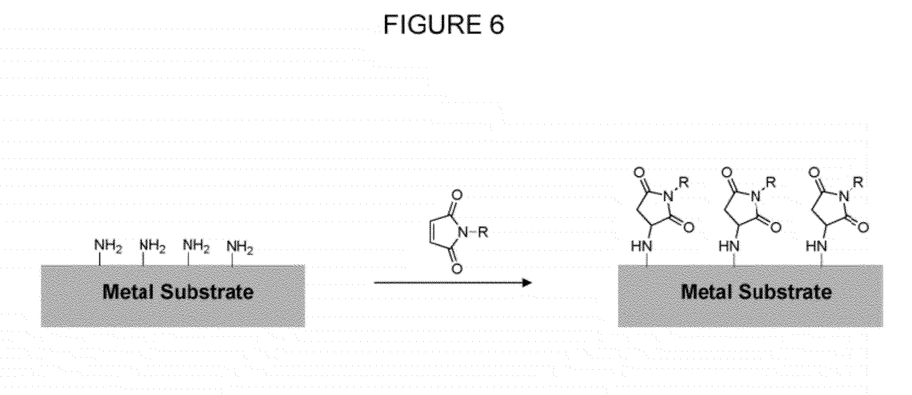

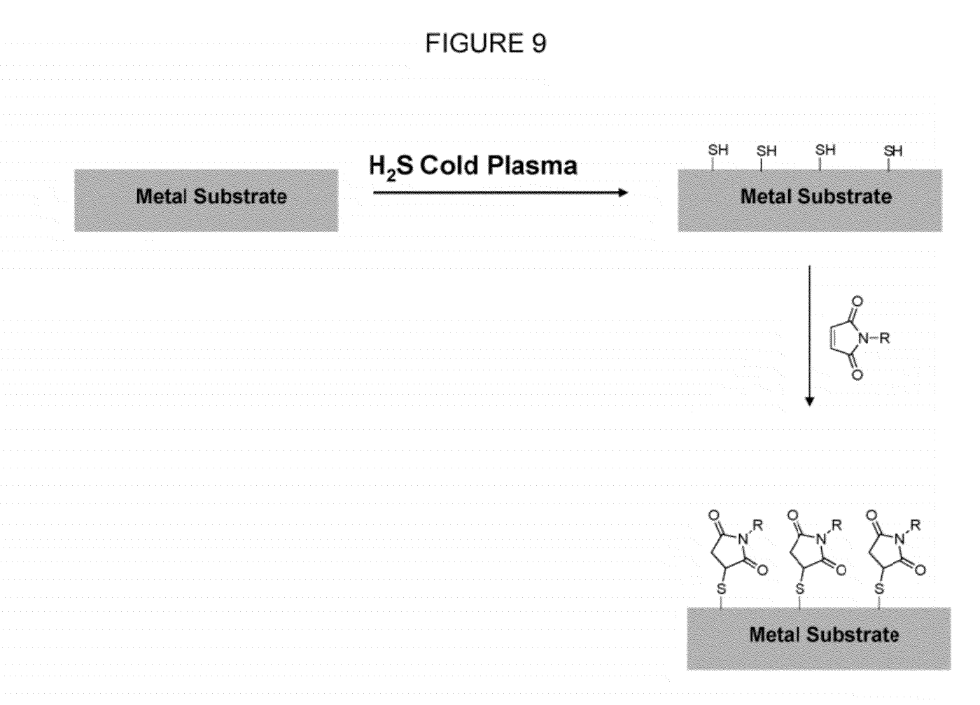

When W is a Michael acceptor and the metal substrate is modified to incorporate a plurality of amine or thiol groups, the coupling at step (c) can occur by Michael addition reaction as depicted in FIG. 6 and FIG. 9.

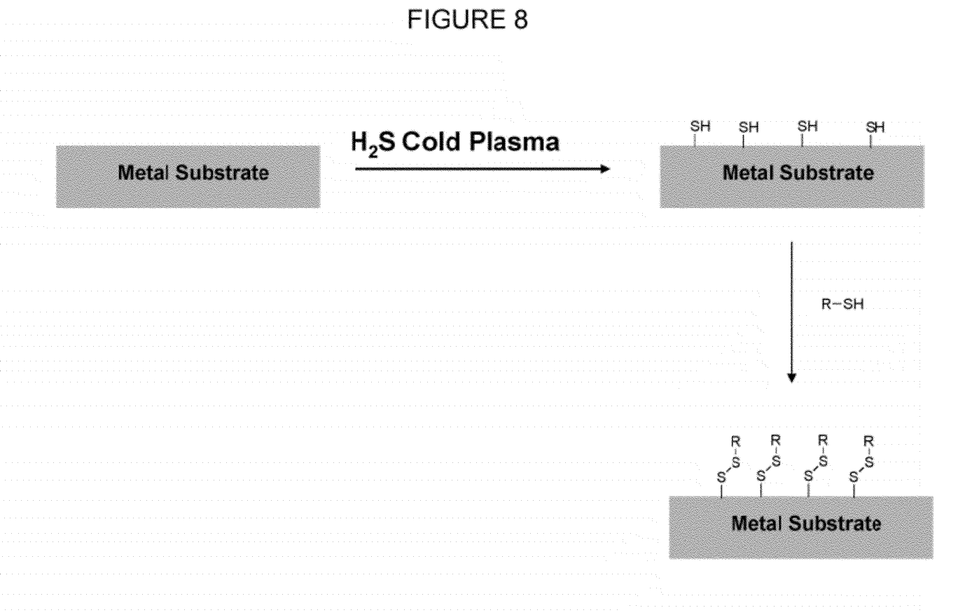

When W is a thiol and the metal substrate is modified to incorporate a plurality of thiol groups, the coupling at step (c) can form disulfide bonds, as depicted in FIG. 8.

It will be appreciated that the R groups depicted in the Figures correspond to compounds in accordance with the present invention.

In certain embodiments, the metal substrate comprises iron, iron alloys, steel, stainless steel, austenitic stainless steel, Type 316 stainless steel, ferritic stainless steel, martensitic stainless steel, duplex stainless steel, cobalt, cobalt alloys, cobalt-chromium alloys, stellite alloys, Vitallium®, titanium, titanium alloys, nickel-titanium alloys, nitinol, or super-alloys.

In other embodiments, R¹ is a synthetic polymer such as linear homopolymers, branched homopolymers, block copolymers, branched block copolymers, star polymers, star copolymers, graft copolymers, hyperbranched copolymers, and dendrimers. In still other embodiments, R¹ is a natural polymer such as oligopeptides, proteins, polynucleic acids (e.g. DNA and RNA), oligosaccharides, and polysaccharides. According to another aspect of the present invention, R¹ is poly(ethylene glycol) (PEG), a heterobifunctional PEG, a branched PEG, heterofunctionalized branched PEG's, PEG-b-PAA-b-PAA block copolymer, PEG-b-PAA-b-PEG block copolymers, PEG-b-polyester-b-PEG block copolymers, PEG-b-PAA block copolymers, [where PAA refers to poly(amino acid)], dextran, heparin, fibronectin, chitosan, amylose, amylopectin, glycogen, xanthan, gellan, pullulun, cellulose, and cellulose acetate.

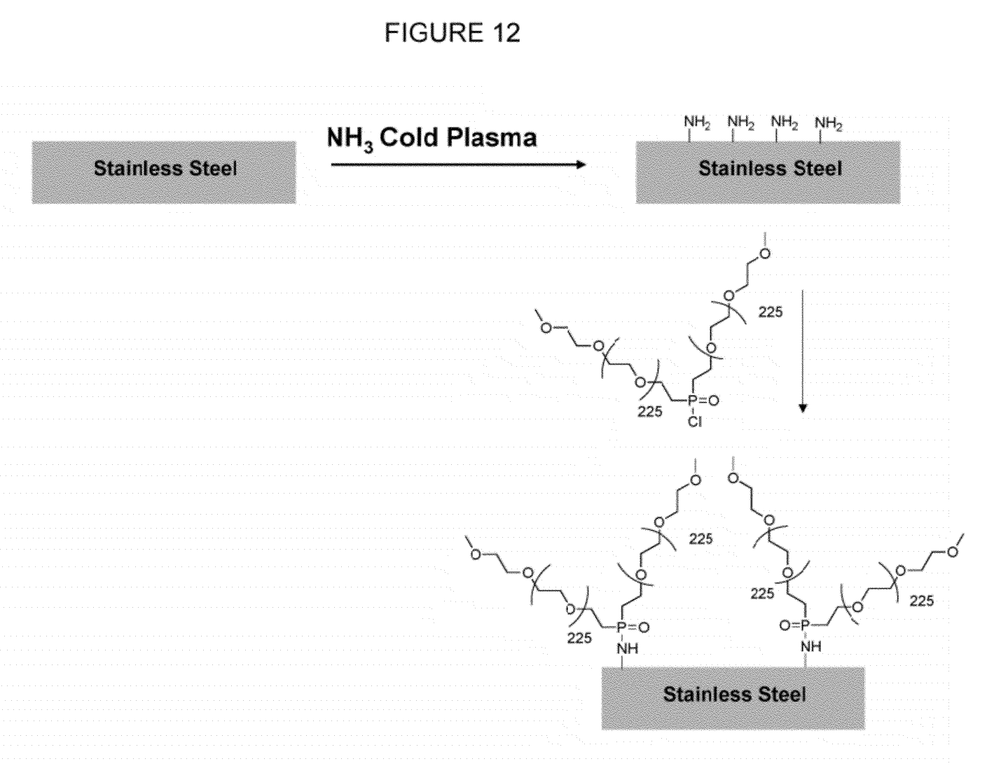

As defined generally above, the R^{a} group of formula I can be a polymer. In certain embodiments, the R^{a} group of formula I is the same as the R¹ group of formula I. An exemplary compound is depicted in FIG. 12. In other embodiments, the R^{a} group of formula I is different from the R¹ group of formula I.

In certain embodiments, R¹ is a small molecule organic group. In other embodiments, R¹ is selected from monosaccharides (e.g., glucose, galactose, fructose) and disaccharides (e.g., sucrose, lactose, maltose), phosphorylcholines, phosoplipids, cyclodextrans, and small molecule drugs.

In other embodiments, the present invention provides a method for preparing a covalently modified metal surface, comprising the steps of:
(a) providing a metal surface having a plurality of amine groups;
(b) providing a compound of formula I:

R¹—W I

wherein:
R¹ is a natural or synthetic polymer or copolymer group or a small molecule organic group;
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
   and
(c) coupling the compound of formula I to one or more of the amine or thiol groups on the metal surface.

### Polymer Groups

As defined generally above, R¹ is a natural or synthetic polymer or copolymer group or a small molecule organic group. In certain embodiments, R¹ is a poly(alkylene oxide) group or a branched poly(alkylene oxide). In other embodiments, R¹ is a poly(ethylene glycol) group (“PEG”). PEG's are well known to one of ordinary skill in the art and include those described in detail in International Patent Application publication number WO2006/047419, U.S. Provisional Patent Application Ser. No. 11/796,385, filed Apr. 27, 2007 and published as WO 2007/27473 on Nov. 8, 2007, and U.S. Provisional Patent Application Ser. No. 11/796,392, filed Apr. 27, 2007 and published as WO 2007/27440 on Nov. 8, 2007, the entirety of each of which is hereby incorporated herein by reference. According to another aspect of the present invention, R¹ is a group of formula II:

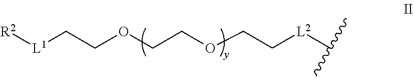

or a salt thereof, wherein:
y is 0-2500;
R² is hydrogen, halogen, NO₂, CN, N₃, —N═C═O, —C(R)═NN(R)₂, —P(O)(OR)₂, —P(O)(X′)₂, a small molecule drug, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

As defined generally above, the y group of formula II is 0-2500. In certain embodiments, the y group of formula II is 0. In certain embodiments, the present invention provides compounds of formula II, as described above, wherein y is about 225. In other embodiments, y is about 10 to about 40. In other embodiments, y is about 40 to about 60. In still other embodiments, y is about 90 to about 150. In still other embodiments, y is about 200 to about 250. In other embodiments, y is about 300 to about 375. In other embodiments, y is about 400 to about 500. In still other embodiments, y is about 650 to about 750. In still other embodiments, y is about 1 to about 10.

In certain embodiments, R² is optionally substituted aliphatic. In other embodiments, R² is an unsubstituted aliphatic. In some embodiments, said R² moiety is an optionally substituted alkyl group. In other embodiments, said R² moiety is an optionally substituted alkynyl or alkenyl group. Such groups include methyl, t-butyl, 5-norbornene-2-yl, octane-5-yl, —C≡CH, —CH₂C≡CH, —CH₂CH₂C≡CH, and —CH₂CH₂CH₂C≡CH. When said R² moiety is a substituted aliphatic group, suitable substituents on R² include any of CN, N₃, NO₂, —CO₂H, —SH, —NH₂, —C(O)H, —NHC(O)R°, —NHC(S)R°, —NHC(O)NR°₂, —NHC(S)NR°₂, —NHC(O)OR°, —NHNHC(O)R°, —NHNHC(O)NR°₂, —NHNHC(O)OR°, —C(O)R°, —C(S)R°, —C(O)OR°, —C(O)SR°, —C(O)OSiR°₃, —OC(O)R°, SC(S)SR°, —SC(O)R°, —C(O)N(R°)₂, —C(S)N(R°)₂, —C(S)SR°, —SC(S)SR°, —OC(O)N(R°)₂, —C(O)NHN(R°)₂, —C(O)N(OR°)R°, —C(O)C(O)R°, —C(O)CH₂C(O)R°, —C(NOR°)R°, —SSR°, —S(O)₂R°, —S(O)₂OR°, —OS(O)₂R°, —S(O)₂N(R°)₂, —S(O)R°, —N(R°)S(O)₂N(R°)₂, —N(R°)S(O)₂R°, —N(OR°)R°, —C(NH)N(R°)₂, —P(O)₂R°, —P(O)(R°)₂, —OP(O)(R°)₂, or —OP(O)(OR°)₂, wherein each R° is as defined herein.

In other embodiments, R² is an aliphatic group optionally substituted with any of Cl, Br, I, F, —NH2, —OH, —SH, —CO₂H, —C(O)H, —C(O)(C₁₋₆ aliphatic), —NHC(O)(C₁₋₆ aliphatic), —NHC(O)NH₂, —NHC(O)NH(C₁₋₆ aliphatic), —NHC(S)NH—, —NHC(S)N(C₁₋₆ aliphatic)₂, —NHC(O)O(C₁₋₆ aliphatic), —NHNH₂, —NHNHC(O)(C₁₋₆ aliphatic), —NHNHC(O)NH₂, —NHNHC(O)NH(C₁₋₆ aliphatic), —NHNHC(O)O(C₁₋₆ aliphatic), —C(O)NH₂, —C(O)NH(C₁₋₆ aliphatic)₂, —C(O)NHNH₂, —C(S)N(C₁₋₆ aliphatic)₂, —OC(O)NH(C₁₋₆ aliphatic), —C(O)C(O)(C₁₋₆ aliphatic), —C(O)CH₂C(O)(C₁₋₆ aliphatic), —S(O)₂(C₁₋₆ aliphatic), —S(O)₂O(C₁₋₆ aliphatic), —OS(O)₂(C₁₋₆ aliphatic), —S(O)₂NH(C₁₋₆ aliphatic), —S(O)(C₁₋₆ aliphatic), —NHS(O)₂NH(C₁₋₆ aliphatic), —NHS(O)₂(C₁₋₆ aliphatic), —P(O)₂(C₁₋₆ aliphatic), —P(O)(C₁₋₆ aliphatic)₂, —OP(O)(C₁₋₆ aliphatic)₂, or —OP(O)(OC₁₋₆ aliphatic)₂.

In certain embodiments, the R² group of formula II is a group suitable for Click chemistry. Click reactions tend to involve high-energy (“spring-loaded”) reagents with well-defined reaction coordinates that give rise to selective bond-forming events of wide scope. Examples include nucleophilic trapping of strained-ring electrophiles (epoxide, aziridines, aziridinium ions, episulfonium ions), certain carbonyl reactivity (e.g., the reaction between aldehydes and hydrazines or hydroxylamines), and several cycloaddition reactions. The azide-alkyne 1,3-dipolar cycloaddition is one such reaction. Click chemistry is known in the art and one of ordinary skill in the art would recognize that certain R² moieties of the present invention are suitable for Click chemistry.

According to one embodiment, the R² group of formula II is an azide-containing group. According to another embodiment, the R² group of formula II is an alkyne-containing group. In certain embodiments, the R² group of formula II has a terminal alkyne moiety. According to another embodiment, the R² group of formula II is an aldehyde-containing group. In certain embodiments, the R² group of formula II has a terminal hydrazine moiety. In other embodiments, the R² group of formula II has a terminal oxyamine moiety. In still other embodiments, the R² group of formula II is a epoxide-containing group. In certain other embodiments, the R² group of formula II has a terminal maleimide moiety.

In other embodiments, R² is an optionally substituted 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur. In certain embodiments, R² is an optionally substituted 5-7 membered saturated or partially unsaturated ring having 0-4 heteroatoms independently selected from nitrogen, oxygen, or sulfur. In other embodiments, R² is an optionally subsituted phenyl ring or a 5-6 membered heteroaryl ring having 1-4 heteroatoms independently selected from nitrogen, oxygen, or sulfur.

In certain embodiments, the R² group of formula II is an optionally substituted aryl group. Examples include optionally substituted phenyl, optionally substituted pyridyl, optionally substituted naphthyl, optionally substituted pyrenyl, optionally substituted triazole, optionally substituted imidazole, optionally substituted phthalimide, optionally substituted tetrazole, optionally substituted furan, and optionally substituted pyran. When said R² moiety is a substituted aryl group, suitable substituents on R² include any of R°, CN, N₃, NO₂, —CH₃, —CH₂N₃, t-butyl, 5-norbornene-2-yl, octane-5-yl, —CH═CH₂, —C≡CH, —CH₂C≡CH, —CH₂CH₂C≡CH, —CH₂CH₂CH₂C≡CH, Cl, Br, I, F, —NH₂, —OH, —SH, —CO₂H, —C(O)H, —CH₂NH₂, —CH₂OH, —CH₂SH, —CH₂CO₂H, —CH₂C(O)H, —C(O)(C₁₋₆ aliphatic), —NHC(O)(C₁₋₆ aliphatic), —NHC(O)NH—, —NHC(O)NH(C₁₋₆ aliphatic), —NHC(S)NH₂, —NHC(S)N(C₁₋₆ aliphatic)₂, —NHC(O)O(C₁₋₆ aliphatic), —NHNH₂, —NHNHC(O)(C₁₋₆ aliphatic), —NHNHC(O)NH₂, —NHNHC(O)NH(C₁₋₆ aliphatic), —NHNHC(O)O(C₁₋₆ aliphatic), —C(O)NH₂, —C(O)NH(C₁₋₆ aliphatic)₂, —C(O)NHNH₂, —C(S)N(C₁₋₆ aliphatic)₂, —OC(O)NH(C₁₋₆ aliphatic), —C(O)C(O)(C₁₋₆ aliphatic), —C(O)CH₂C(O)(C₁₋₆ aliphatic), —S(O)₂(C₁₋₆ aliphatic), —S(O)₂O(C₁₋₆ aliphatic), —OS(O)₂(C₁₋₆ aliphatic), —S(O)₂NH(C₁₋₆ aliphatic), —S(O)(C₁₋₆ aliphatic), —NHS(O)₂NH(C₁₋₆ aliphatic), —NHS(O)₂(C₁₋₆ aliphatic), —P(O)₂(C₁₋₆ aliphatic), —P(O)(C₁₋₆ aliphatic)₂, —OP(O)(C₁₋₆ aliphatic)₂, or —OP(O)(OC₁₋₆ aliphatic)₂.

Suitable substitutents on R² further include bis-(4-ethynyl-benzyl)-amino, dipropargylamino, di-hex-5 -ynyl-amino, di-pent-4-ynyl-amino, di-but-3 -ynyl-amino, propargyloxy, hex-5-ynyloxy, pent-4-ynyloxy, di-but-3-ynyloxy, 2-hex-5-ynyloxy-ethyldisulfanyl, 2-pent-4-ynyloxy-ethyldisulfanyl, 2-but-3-ynyloxy-ethyldisulfanyl, 2-propargyloxy-ethyldisulfanyl, bis-benzyloxy-methyl, [1,3]dioxolan-2-yl, and [1,3]dioxan-2-yl.

In other embodiments, R² is hydrogen.

According to one embodiment, R² is methyl.

In certain embodiments, R² is N₃.

In other embodiments, R² is an epoxide ring.

In certain embodiments, the R² group of formula II is a crown ether. Examples of such crown ethers include 12-crown-4, 15-crown-5, and 18-crown-6.

In still other embodiments, R² is a detectable moiety. Detectable moieties are known in the art and include those described herein. According to one aspect of the invention, the R² group of formula II is a fluorescent moiety. Such fluorescent moieties are well known in the art and include coumarins, quinolones, benzoisoquinolones, hostasol, and Rhodamine dyes, to name but a few. Exemplary fluorescent moieties of R² include anthracen-9-yl, pyren-4-yl, 9-H-carbazol-9-yl, the carboxylate of rhodamine B, and the carboxylate of coumarin 343. In certain embodiments, R² is a detectable moiety selected from:

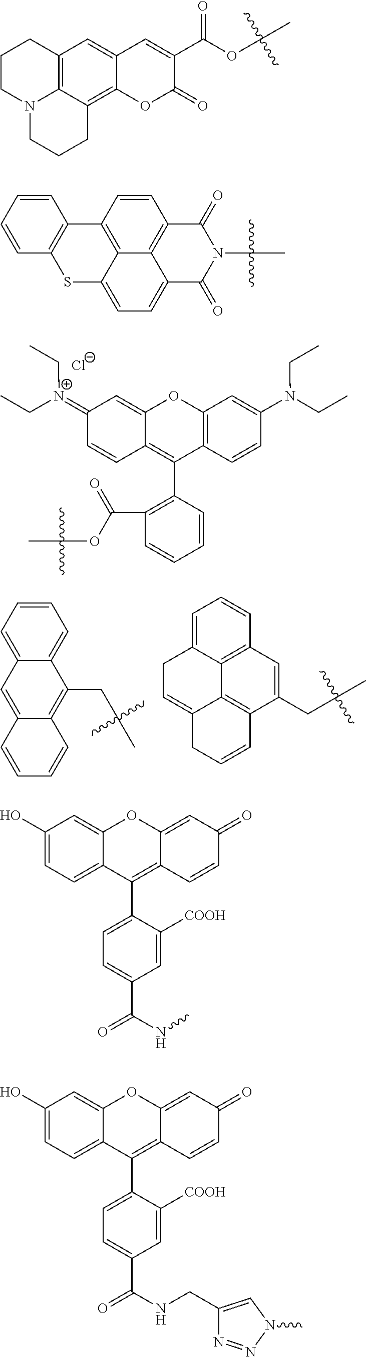

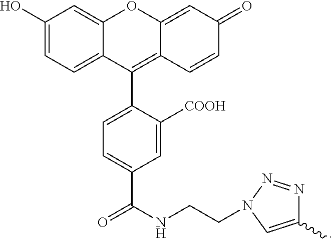

In certain embodiments, R² is —P(O)(OR)₂, or —P(O)(halogen)₂. According to one aspect, the present invention provides a compound of formula II, wherein R² is —P(O)(OH)₂. According to another aspect, the present invention provides a compound of formula II, wherein R² is —P(O)(Cl)₂. One of ordinary skill in the art would recognize that when R² is —P(O)(OR)₂, or —P(O)(halogen)₂, the R² group is also capable of forming a covalent bond with the hydrophilic metal surface thus forming a “looped” attachment.

As defined generally above, the L¹ group of formula II is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, L¹ is a valence bond. In other embodiments, L¹ is a bivalent, saturated C₁₋₁₂ alkylene chain, wherein 0-6 methylene units of L¹ are independently replaced by -Cy-, —O—, —NH—, —S—, —OC(O)—, —C(O)O—, —C(O)—, —C(O)NH—, or —NHC(O)—, wherein each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In still other embodiments, L¹ is a bivalent, saturated C₁₋₆ alkylene chain, wherein 0-3 methylene units of L¹ are independently replaced by -Cy-, —O—, —NH—, —S—, —OC(O)—, —C(O)O—, —C(O)—, —C(O)NH—, or —NHC(O)—,

In certain embodiments, L¹ is -Cy- (i.e. a C₁ alkylene chain wherein the methylene unit is replaced by -Cy-), wherein -Cy- is an optionally substituted 3-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

In certain embodiments, the L¹ group of formula II is —O—, —S—, —NH—, or —C(O)O—. In other embodiments, the L¹ group of formula II is -Cy-, —C(O)—, —C(O)NH—, —NHC(O)—, —NH—O—, or —O-Cy-CH₂NH—O—. In still other embodiments, the L¹ group of formula II is any of —OCH₂—, —OCH₂C(O)—, —OCH₂CH₂C(O)—, —OCH₂CH₂O—, —OCH₂CH₂S—, —OCH₂CH₂C(O)O—, —OCH₂CH₂NH—, —OCH₂CH₂NHC(O)—, —OCH₂CH₂C(O)NH—, and —NHC(O)CH₂CH₂C(O)O—. According to another aspect, the L¹ group of formula II is any of _OCH₂CH₂NHC(O)CH₂CH₂C(O)O—, —OCH₂CH₂NHC(O)CH₂OCH₂C(O)O—, —OCH₂CH₂NHC(O)CH₂OCH₂C(O)NH—, —CH₂C(O)NH—, —CH₂C(O)NHNH—, or —OCH₂CH₂NHNH—.

As defined generally above, the R² group of formula II is, inter alia, a mono-protected amine, a di-protected amine, a protected aldehyde, a protected hydroxyl, a protected carboxylic acid, or a protected thiol. Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate(trimethylacetate), crotonate, 4-methoxy-crotonate, benzoate, p-benylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

As defined generally above, the L² group of formula II is L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, L² is a valence bond. In other embodiments, L² is a bivalent, saturated C₁₋₁₂ alkylene chain, wherein 0-6 methylene units of L² are independently replaced by -Cy-, or —O—, —NH—, wherein each -Cy- is independently an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In still other embodiments, L² is a bivalent, saturated C₁₋₆ alkylene chain, wherein 0-2 methylene units of L² are independently replaced by -Cy-.

In certain embodiments, L² is -Cy- (i.e., a C₁ alkylene chain wherein the methylene unit is replaced by -Cy-), wherein -Cy- is an optionally substituted 3-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

In certain embodiments, the L² group of formula II is —O—, —S—, —NH—, or —C(O)—. In still other embodiments, the L² group of formula II is any of —OCH₂—, —OCH₂C(O)—, —OCH₂CH₂C(O)—, —OCH₂CH₂O—, or —OCH₂CH₂S—. In other embodiments, the L² group of formula II is —OC(O)CH₂CH₂CH₂CH₂—, —OCH₂CH₂—, —NHC(O)CH₂CH₂—, —NHC(O)CH₂CH₂CH₂—, —OC(O)CH₂CH₂CH₂—, —O-Cy-, —O-Cy-CH₂—, —O-Cy-NH—, —O-Cy-S—, —O-Cy-C(O)—, or —O-Cy-C(O)O-Cy-. In certain embodiments, the L² group of formula II is —O—.

In certain embodiments, the present invention provides a method for preparing a covalently modified metal surface, comprising the steps of:
(a) modifying a metal substrate to incorporate thereon a plurality of amine or thiol groups;
(b) providing a compound of formula II-a:

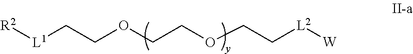

or a salt thereof, wherein:
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
y is 0-2500;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.
      and
(c) coupling the compound of formula II-a to one or more of the amine or thiol groups on the metal surface.

Each of the R², L¹, y, and L² groups of formula II-a are as described in classes and subclasses for compounds of formula II, both singly and in combination.

In other embodiments, the present invention provides a method for preparing a covalently modified metal surface, comprising the steps of:
(a) modifying a metal substrate to incorporate thereon a plurality of amine or thiol groups;
(b) providing a compound of formula II-b:

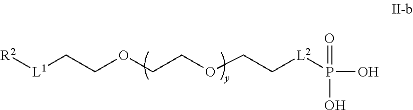

or a salt thereof, wherein:
y is 10-2500;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.
      and
(c) coupling the compound of formula II-b to one or more of the amine or thiol groups on the metal surface by dehydration reaction.

Each of the R², L¹, y, and L² groups of formula II-b are as described in classes and subclasses for compounds of formula II, both singly and in combination.

In other embodiments, the present invention provides a method for preparing a covalently modified metal surface, comprising the steps of:
(a) modifying a metal substrate to incorporate thereon a plurality of amine or thiol groups;
(b) providing a compound of formula II-c:

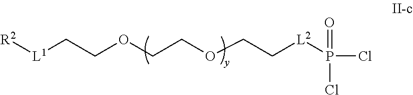

or a salt thereof, wherein:
y is 10-2500;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.
      and
(c) coupling the compound of formula II-c to one or more of the amine or thiol groups on the metal surface by condensation reaction.

Each of the R², L¹, y, and L² groups of formula II-c are as described in classes and subclasses for compounds of formula II, both singly and in combination.

In certain embodiments, the R¹ group of formula I is a group of formula II-d:

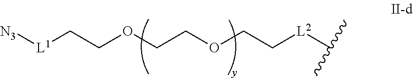

or a salt thereof, wherein:
y is 0-2500;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

Each of the L¹, y, and L² groups of formula II-d are as described in classes and subclasses for compounds of formula II, both singly and in combination.

In certain embodiments, the present invention provides a method for preparing a covalently modified metal surface, comprising the steps of:
(a) modifying a metal substrate to incorporate thereon a plurality of amine or thiol groups;
(b) providing a compound of formula II-e:

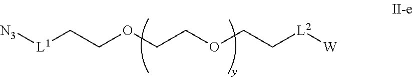

or a salt thereof, wherein:
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
y is 0-2500;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur,
      and
(c) coupling the compound of formula II-e to one or more of the amine or thiol groups on the metal surface.

Each of the L¹, y, W, and L² groups of formula II-e are as described in classes and subclasses for compounds of formulae I and II, both singly and in combination.

In other embodiments, the present invention provides a method for preparing a covalently modified metal surface, comprising the steps of:
(a) modifying a metal substrate to incorporate thereon a plurality of amine or thiol groups;
(b) providing a compound of formula II-e; and
(c) coupling the compound of formula II-e to one or more of the amine or thiol groups on the metal surface, further comprising the step of coupling the azide-terminal end to a suitable group via Click chemistry.

In certain embodiments, step (c) includes the step of coupling the azide-terminal end to a Click-ready biomolecule conjugated. In certain embodiments, the present invention provides a method of incorporating a targeting group, a therapeutic or a therapeutic carrier such as a protein, a cell, a virus particle, a plasmid, an oligopeptide, an oligonucleotide (e.g. siRNA, miRNA, aptamer), small molecule drug, a liposome, a polymersome, a polymer microshere, or a lipid emulsion onto a covalently modified metal substrate as described generally above and in classes and subclasses defined above and herein. It will be appreciated that the R¹ group of formula I can incorporate an acetylene moiety and that such a moiety can also be used in coupling a provided metal surface to a biomolecule via Click chemistry.

In certain embodiments, a targeting group is coupled to a covalently modified metal surface via Click chemistry. In some embodiments, a small molecule drug is coupled to a covalently modified metal surface via Click chemistry.

In other embodiments, the R¹ group of formula I is a copolymer group. Multiblock copolymers of the present invention are prepared by methods known to one of ordinary skill in the art and those described in detail in United States patent publication US 20060172914, published Aug. 3, 2006, the entirety of which is hereby incorporated herein by reference. According to another aspect, R¹ is a block copolymer group of formula III:

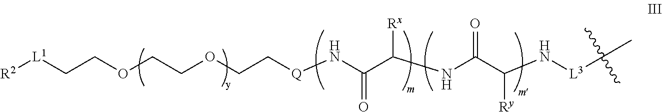

wherein:
y is 1-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L³ is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, the m′ group of formula III is 1-1000. In certain embodiments, the m′ group of formula III is 0. In other embodiments, m′ is 1-1000. According to other embodiments, m and m′ are independently 10 to 100 repeat units. In still other embodiments, m is 1-20 repeat units and m′ is 10-50 repeat units.

As defined generally above, the Q group of formula III is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur. In certain embodiments, Q is a valence bond. In other embodiments, Q is a bivalent, saturated C₁₋₁₂ alkylene chain, wherein 0-6 methylene units of Q are independently replaced by -Cy-, —O—, —NH—, —S—, —OC(O)—, —C(O)O—, or —C(O)—, wherein -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In certain embodiments, Q is -Cy- (i.e. a C₁ alkylene chain wherein the methylene unit is replaced by -Cy-), wherein -Cy- is an optionally substituted 5-8 membered bivalent, saturated, partially unsaturated, or aryl ring having 0-4 heteroatoms independently selected from nitrogen, oxygen, or sulfur. According to one aspect of the present invention, -Cy- is an optionally substituted bivalent aryl group. According to another aspect of the present invention, -Cy- is an optionally substituted bivalent phenyl group. In other embodiments, -Cy- is an optionally substituted 5-8 membered bivalent, saturated carbocyclic ring. In still other embodiments, -Cy- is an optionally substituted 5-8 membered bivalent, saturated heterocyclic ring having 1-2 heteroatoms independently selected from nitrogen, oxygen, or sulfur. Exemplary -Cy- groups include bivalent rings selected from phenyl, pyridyl, pyrimidinyl, cyclohexyl, cyclopentyl, or cyclopropyl.

In certain embodiments, R^{x} is a crosslinkable amino acid side-chain group and R^{y} is a hydrophobic amino acid side-chain group. Such crosslinkable amino acid side-chain groups include tyrosine, serine, cysteine, threonine, aspartic acid (also known as aspartate, when charged), glutamic acid (also known as glutamate, when charged), asparagine, histidine, lysine, arginine, and glutamine. Such hydrophobic amino acid side-chain groups include a suitably protected tyrosine side-chain, a suitably protected serine side-chain, a suitably protected threonine side-chain, phenylalanine, alanine, valine, leucine, tryptophan, proline, benzyl and alkyl glutamates, or benzyl and alkyl aspartates or mixtures thereof. In other embodiments, R^{y} is an ionic amino acid side-chain group. Such ionic amino acid side chain groups includes a lysine side-chain, arginine side-chain, or a suitably protected lysine or arginine side-chain, an aspartic acid side chain, glutamic acid side-chain, or a suitably protected aspartic acid or glutamic acid side-chain. One of ordinary skill in the art would recognize that protection of a polar or hydrophilic amino acid side-chain can render that amino acid nonpolar. For example, a suitably protected tyrosine hydroxyl group can render that tyrosine nonpolar and hydrophobic by virtue of protecting the hydroxyl group. Suitable protecting groups for the hydroxyl, amino, and thiol, and carboylate functional groups of R^{x} and R^{y} are as described herein.

In other embodiments, R^{y} comprises a mixture of hydrophobic and hydrophilic amino acid side-chain groups such that the overall poly(amino acid) block comprising R^{y} is hydrophobic. Such mixtures of amino acid side-chain groups include phenylalanine/tyrosine, phenalanine/serine, leucine/tyrosine, and the like. According to another embodiment, R^{y} is a hydrophobic amino acid side-chain group selected from phenylalanine, alanine, or leucine, and one or more of tyrosine, serine, or threonine.

As defined above, R^{x} is a natural or unnatural amino acid side-chain group capable of forming cross-links. It will be appreciated that a variety of amino acid side-chain functional groups are capable of such cross-linking, including, but not limited to, carboxylate, hydroxyl, thiol, and amino groups. Examples of R^{x} moieties having functional groups capable of forming cross-links include a glutamic acid side-chain, —CH₂C(O)CH, an aspartic acid side-chain, —CH₂CH₂C(O)OH, a cystein side-chain, —CH₂SH, a serine side-chain, —CH₂OH, an aldehyde containing side-chain, —CH₂C(O)H, a lysine side-chain, —(CH₂)₄NH₂, an arginine side-chain, —(CH₂)₃NHC(═NH)NH₂, a histidine side-chain, —CH₂-imidazol-4-yl.

In certain embodiments, the present invention provides a method for preparing a covalently modified metal surface, comprising the steps of:
(a) modifying a metal substrate to incorporate thereon a plurality of amine or thiol groups;
(b) providing a compound of formula III-a:

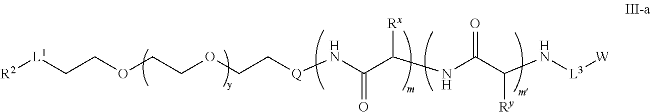

wherein:
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
y is 1-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L³ is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
      and
(c) coupling the compound of formula III-a to one or more of the amine or thiol groups on the metal surface.

Each of the R², L¹, y, m, m′, Q, R^{x}, R^{y}, and L³ groups of formula III-a are as described in classes and subclasses for compounds of formulae II and III, both singly and in combination.

In certain embodiments, the present invention provides a method for preparing a covalently modified metal surface, comprising the steps of:
(a) modifying a metal substrate to incorporate thereon a plurality of amine or thiol groups;
(b) providing a compound of formula III-b:

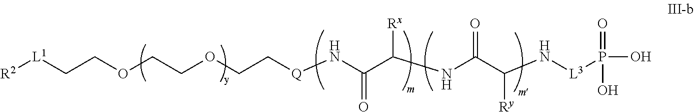

wherein:
y is 1-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L³ is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
      and
(c) coupling the compound of formula III-b to one or more of the amine or thiol groups on the metal surface by dehydration reaction.

Each of the R², L¹, y, m, m′, Q, R^{x}, R^{y}, and L³ groups of formula III-b are as described in classes and subclasses for compounds of formula III, both singly and in combination.

In certain embodiments, the present invention provides a method for preparing a covalently modified metal surface, comprising the steps of:
(a) modifying a metal substrate to incorporate thereon a plurality of amine or thiol groups;
(b) providing a compound of formula III-c:

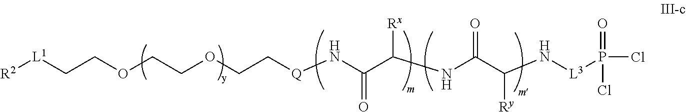

wherein:
y is 10-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L³ is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
      and
(c) coupling the compound of formula III-c to one or more of the amine or thiol groups on the metal surface by dehydration reaction.

Each of the R², L¹, y, m, m′, Q, R^{x}, R^{y}, and L³ groups of formula III-c are as described in classes and subclasses for compounds of formula III, both singly and in combination.

In other embodiments, R¹ is a block copolymer group of formula IV:

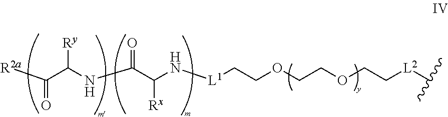

wherein:
y is 10-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
   -Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³; and
each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
   two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

Each of the R², L¹, y, m, m′, Q, R^{x}, R^{y}, and L² groups of formula IV are as described in classes and subclasses for compounds of formulae II and III, both singly and in combination.

As defined generally above, the R^{2a} group of formula IV is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³, wherein each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

In certain embodiments, the R^{2a} group of formula IV is —NHR³ or —N(R³)₂ wherein each R³ is an optionally substituted aliphatic group. One exemplary R³ group is 5-norbornen-2-yl-methyl. According to yet another aspect of the present invention, the R^{2a} group of formula IV is —NHR³ wherein R³ is a C₁₋₆ aliphatic group substituted with N₃. Examples include —CH₂N₃. In some embodiments, R³ is an optionally substituted C₁₋₆ alkyl group. Examples include methyl, ethyl, propyl, butyl, pentyl, hexyl, 2-(tetrahydropyran-2-yloxy)ethyl, pyridin-2-yldisulfanylmethyl, methyldisulfanylmethyl, (4-acetylenylphenyl)methyl, 3-(methoxycarbonyl)-prop-2-ynyl, methoxycarbonylmethyl, 2-(N-methyl-N-(4-acetylenylphenyl)carbonylamino)-ethyl, 2-phthalimidoethyl, 4-bromobenzyl, 4-chlorobenzyl, 4-fluorobenzyl, 4-iodobenzyl, 4-propargyloxybenzyl, 2-nitrobenzyl, 4-(bis-4-acetylenylbenzyl)aminomethyl-benzyl, 4-propargyloxy-benzyl, 4-dipropargylamino-benzyl, 4-(2-propargyloxy-ethyldisulfanyl)benzyl, 2-propargyloxy-ethyl, 2-prop argyldisulfanyl-ethyl, 4-propargyloxy-butyl, 2-(N-methyl-N-propargylamino)ethyl, and 2-(2-dipropargylaminoethoxy)-ethyl. In other embodiments, R³ is an optionally substituted C₂₋₆ alkenyl group. Examples include vinyl, allyl, crotyl, 2-propenyl, and but-3-enyl. When R³ group is a substituted aliphatic group, suitable substituents on R³ include N₃, CN, and halogen. In certain embodiments, R³ is —CH₂CN, —CH₂CH₂CN, —CH₂CH(OCH₃)₂, 4-(bisbenzyloxymethyl)phenylmethyl, and the like.

According to another aspect of the present invention, the R^{2a} group of formula IV is —NHR³ wherein R³ is an optionally substituted C₂₋₆ alkynyl group. Examples include —CC≡CH, —CH₂C≡CH, —CH₂C≡CCH₃, and —CH₂CH₂C≡CH.

In certain embodiments, the R^{2a} group of formula IV is —NHR³ wherein R³ is an optionally substituted 5-8-membered aryl ring. In certain embodiments, R³ is optionally substituted phenyl or optionally substituted pyridyl. Examples include phenyl, 4-t-butoxycarbonylaminophenyl, 4-azidomethylphenyl, 4-propargyloxyphenyl, 2-pyridyl, 3-pyridyl, and 4-pyridyl. In certain embodiments, R^{2a} is 4-t-butoxycarbonylaminophenylamino, 4-azidomethylphenamino, or 4-prop argyloxyphenylamino.

In certain embodiments, the R^{2a} group of formula IV is —NHR³ wherein R³ is an optionally substituted phenyl ring. Suitable substituents on the R³ phenyl ring include halogen; —(CH₂)₀₋₄R°; —(CH₂)₀₋₄OR°; —(CH₂)₀₋₄CH(OR°)₂; —(CH₂)₀₋₄SR°; —(CH₂)₀₋₄Ph, which may be substituted with R°; —(CH₂)₀₋₄O(CH₂)₀₋₁Ph which may be substituted with R°; —CH═CHPh, which may be substituted with R°; —NO₂; —CN; —N₃; —(CH₂)₀₋₄N(R°)₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°)C(O))R°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —(CH₂)₀₋₄SC(O)R°; —(CH₂)₀₋₄C(O)NR°₂; —C(S)NR°₂; —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; SiR°₃; wherein each independent occurrence of R° is as defined herein supra. In other embodiments, the R^{2a} group of formula IV is —NHR³ wherein R³ is phenyl substituted with one or more optionally substituted C₁₋₆ aliphatic groups. In still other embodiments, R³ is phenyl substituted with vinyl, allyl, acetylenyl, —CH₂N₃, —CH₂CH₂N₃, —CH₂C≡CCH₃, or —CH₂C≡CH.

In certain embodiments, the R^{2a} group of formula IV is —NHR³ wherein R³ is phenyl substituted with N₃, N(R°)₂, CO₂R°, or C(O)R° wherein each R° is independently as defined herein supra.

In certain embodiments, the R^{2a} group of formula IV is —N(R³)₂ wherein each R³ is independently an optionally substituted group selected from aliphatic, phenyl, naphthyl, a 5-6 membered aryl ring having 1-4 heteroatoms independently selected from nitrogen, oxygen, or sulfur, or a 8-10 membered bicyclic aryl ring having 1-5 heteroatoms independently selected from nitrogen, oxygen, or sulfur, or a detectable moiety.

In other embodiments, the R^{2a} group of formula IV is —N(R³)₂ wherein the two R³ groups are taken together with said nitrogen atom to form an optionally substituted 4-7 membered saturated, partially unsaturated, or aryl ring having 1-4 heteroatoms independently selected from nitrogen, oxygen, or sulfur. According to another embodiment, the two R³ groups are taken together to form a 5-6-membered saturated or partially unsaturated ring having one nitrogen wherein said ring is substituted with one or two oxo groups. Such R^{2a} groups include, but are not limited to, phthalimide, maleimide and succinimide.

In certain embodiments, the R^{2a} group of formula IV is a mono-protected or di-protected amino group. In certain embodiments R^{2a} is a mono-protected amine. In certain embodiments R^{2a} is a mono-protected amine selected from aralkylamines, carbamates, allyl amines, or amides. Examplary mono-protected amino moieties include t-butyloxycarbonylamino, ethyloxycarbonylamino, methyloxycarbonylamino, trichloroethyloxy-carbonylamino, allyloxycarbonylamino, benzyloxocarbonylamino, allylamino, benzylamino, fluorenylmethylcarbonyl, formamido, acetamido, chloroacetamido, dichloroacetamido, trichloroacetamido, phenylacetamido, trifluoroacetamido, benzamido, and t-butyldiphenylsilylamino. In other embodiments R^{2a} is a di-protected amine. Exemplary di-protected amino moieties include di-benzylamino, di-allylamino, phthalimide, maleimido, succinimido, pyrrolo, 2,2,5,5-tetramethyl-[1,2,5]azadisilolidino, and azido. In certain embodiments, the R^{2a} moiety is phthalimido. In other embodiments, the R^{2a} moiety is mono- or di-benzylamino or mono- or di-allylamino.

In certain embodiments, the R^{2a} group of formula IV comprises a group suitable for Click chemistry. One of ordinary skill in the art would recognize that certain R^{2a} groups of the present invention are suitable for Click chemistry.

Compounds of formula IV having R^{2a} groups comprising groups suitable for Click chemistry are useful for conjugating said compounds to biological systems such as proteins, viruses, and cells, to name but a few. Thus, another embodiment of the present invention provides a method of conjugating the R^{2a} group of a compound of formula IV to a macromolecule via Click chemistry. Yet another embodiment of the present invention provides a macromolecule conjugated to a compound of formula IV via the R^{2a} group.

According to one embodiment, the R^{2a} group of formula IV is an azide-containing group. According to another embodiment, the R^{2a} group of formula IV is an alkyne-containing group.

In certain embodiments, the R^{2a} group of formula IV has a terminal alkyne moiety. In other embodiments, the R^{2a} group of formula IV is an alkyne-containing moiety having an electron withdrawing group. Accordingly, in such embodiments, the R^{2a} group of formula IV is

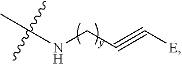

wherein E is an electron withdrawing group and y is 0-6. Such electron withdrawing groups are known to one of ordinary skill in the art. In certain embodiments, E is an ester. In other embodiments, the R^{2a} group of formula IV is

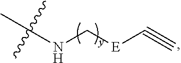

wherein E is an electron withdrawing group, such as a —C(O)O— group and y is 0-6.

According to another embodiment, the present invention provides methods utilizing compounds of formula IV, as described above, wherein said compounds have a polydispersity index (“PDI”) of about 1.0 to about 1.2. According to another embodiment, the present invention provides compounds of formula IV, as described above, wherein said compound has a polydispersity index (“PDI”) of about 1.03 to about 1.15. According to yet another embodiment, the present invention provides compounds of formula IV, as described above, wherein said compound has a polydispersity index (“PDI”) of about 1.10 to about 1.12. According to other embodiments, the present invention provides compounds of formula IV having a PDI of less than about 1.10.

In certain embodiments, the present invention utilizes compounds of formula IV, as described above, wherein n is about 225. In other embodiments, n is about 200 to about 300. In still other embodiments, n is about 200 to about 250. In still other embodiments, n is about 100 to about 150. In still other embodiments, n is about 400 to about 500.

Exemplary R^{2a} groups of formula IV are set forth in Table 1, below.

TABLE 1
Representative R^{2a} Groups

| | |
|---|---|
| 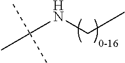 | i |
| 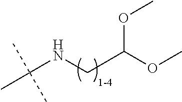 | ii |
| 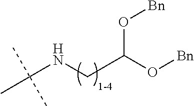 | iii |
| 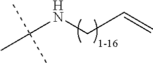 | iv |
| 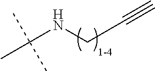 | v |
| 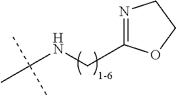 | vi |
| 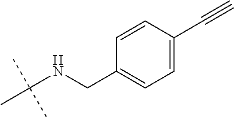 | vii |
| 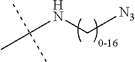 | viii |
| 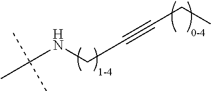 | ix |
| 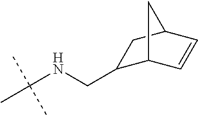 | x |
| 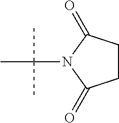 | x |
| 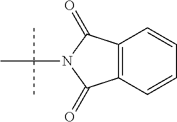 | xi |
| 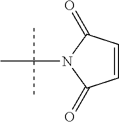 | xii |
| 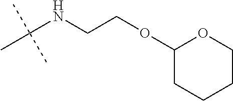 | xiii |
| 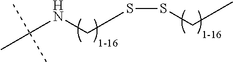 | xiv |
| 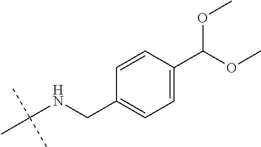 | xv |
| 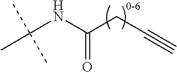 | xvi |
| 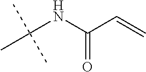 | xvii |
| 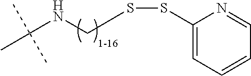 | xviii |
| 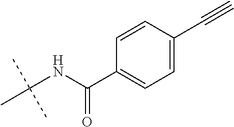 | xix |
| 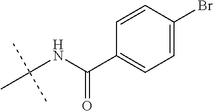 | xx |
| 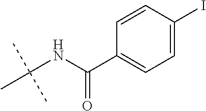 | xxi |
| 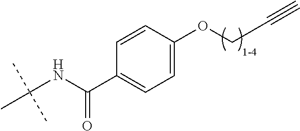 | xxii |
| 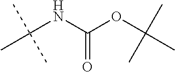 | xxiii |
| 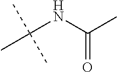 | xxiv |
| 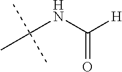 | xxv |
| 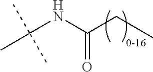 | xxvi |
| 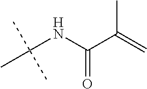 | xxvii |
| 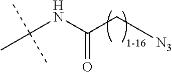 | xxviii |
| 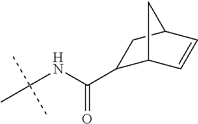 | xxix |
| 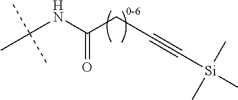 | xxx |
| 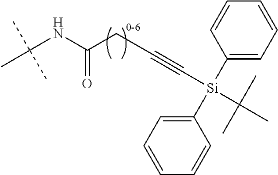 | xxxi |
| 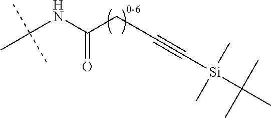 | xxxii |
| 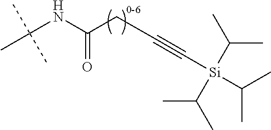 | xxxiii |
| 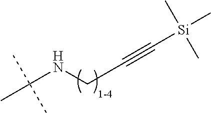 | xxxiv |
| 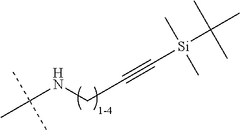 | xxxv |
| 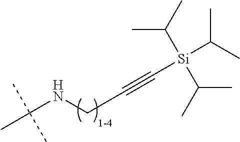 | xxxvi |
| 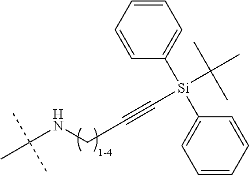 | xxxvii |
| 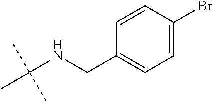 | xxxviii |
| 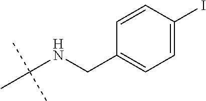 | xxxix |
| 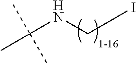 | xl |
| 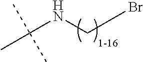 | xli |
| 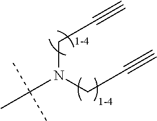 | xlii |
| 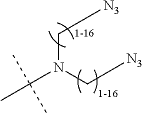 | xliii |
| 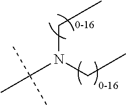 | xliv |
| 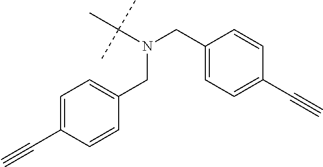 | xlv |
| 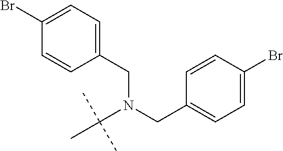 | xlvi |
| 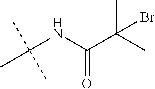 | xlvii |

In certain embodiments, the R^{2a} group of formula IV is selected from any of those R^{2a} groups depicted in Table 1, supra. In other embodiments, the R^{2a} group of formula IV is group v, viii, xvi, xix, xxii, xxx, xxxi, xxxii, xxxiii, xxxiv, xxxv, xxxvi, xxxvii, or xlii. In yet other embodiments, the R^{2a} group of formula IV is xv, xviii, xx, xxi, xxxviii, or xxxix.

### Small Molecule Organic Groups

As defined generally above, R¹ is, inter alia, a small molecule organic group. In certain embodiments, R¹ is selected from monosaccharides (e.g., glucose, galactose, or fructose) disaccharides (e.g., sucrose, lactose, or maltose), phosphorylcholines, phosoplipids, cyclodextran, small molecule drugs, optionally substituted aliphatic groups, optionally substituted cyclic groups, detectable moieties, and the like.

In certain embodiments, R¹ is a group of formula V:

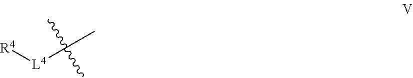

R⁴ is hydrogen, halogen, NO₂, CN, N₃, —N═C═O, —C(R)═NN(R)₂, —P(O)(OR)₂, —P(O)(X″)₂, a 9-30 membered crown ether, a small molecule drug, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X″ is independently halogen;
each R is independently hydrogen or an optionally substituted aliphatic group;
L⁴ is a valence bond or a bivalent, saturated or unsaturated, straight or branched C₁₋₁₂ alkylene chain, wherein 0-6 methylene units of L⁴ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
   each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

In other embodiments, the R¹ group of formula I or the R⁴ group of formula V is a small molecule drug. For formula I, it is contemplated that the small molecule drug is either bonded directly to W or through a linker group L⁵, wherein L⁵ is as defined for the L² group of formula II and in classes and subclasses described for L² herein. In certain embodiments, the linker group L⁴ or L⁵ is a hydrolytically cleavable linker group. It will be appreciated that when the R¹ group of formula I or the R⁴ group of formula V is a small molecule drug and the corresponding L⁴ or L⁵ linker group is a hydrolytically cleavable linker group, then the small molecule drug can be slowly released by the metal surface covalently modified therewith, upon, for example, implantation into a patient. Such hydrolytically cleavable linker groups are well known to one or ordinary skill in the art.

In certain embodiments, the small molecule drug is a member of the taxane family of anti-tubulin agents. In other embodiments, the small molecule drug is paclitaxel. In still other embodiments, the small molecule drug is docetaxel. In certain embodiments, the small molecule drug is a member of the anthracyline family of cytotoxic agents. In other embodiments, the small molecule drug is doxorubicin. In still other embodiments, the small molecule drug is daunorubicin. In still other embodiments, the small molecule drug is epirubicin.

In other embodiments, the R¹ group of formula I or the R⁴ group of formula V is an antithrombogenic oligopeptide. Such antithrombogenic oligopeptide are well known in the art and include sequences such as Cys-Pro-Arg, Cys-(L)Phe-Pro-Arg, and/or Cys-(D)Phe-Pro-Arg.

It will also be appreciated that the R² group of formula II can be an antithromobgenic oligopeptide, such as those name above, attached to PEG via a hydrolytically stable linkage.

In other embodiments, the R¹ group of formula I or the R⁴ group of formula V is a cell-binding oligopeptide. Cell-binding oligopeptides are well known in the art and include αᵥβ₃ and αᵥβ₅ integrin binding peptide sequences such as those containing the Arg-Gly-Asp (RGD) and Asn-Gly-Arg (NGR) oligopeptide sequences. In one embodiment, the R¹ group of formula I or the R⁴ group of formula V is the cell-binding oligopeptide GRGDS. In another embodiment, the oligopeptide sequence is a cyclic RGD sequence such as c(RGDfK).

It will also be appreciated that the R² group of formula II can be a cell-binding oligopeptide, such as those name above, attached to PEG via a hydrolytically stable linkage

It will also be appreciated that when the R² group of formula II is a small molecule drug attached to the PEG via a hydrolytically cleavable linker group, then that small molecule drug is slowly released by the metal surface covalently modified therewith leaving the PEGylated metal surface. In certain embodiments, the small molecule drug connected to PEG via a hydrolytically cleavable linker is a member of the taxane family of anti-tubulin agents. In other embodiments, the small molecule drug connected to PEG via a hydrolytically cleavable linker is paclitaxel. In still other embodiments, the small molecule drug connected to PEG via a hydrolytically cleavable linker is docetaxel.

It will also be appreciated that when the polymers of formula III are used for surface modification, a hydrophobic small molecule drug can be encapsulated in the hydrophobic region of polymer layer. Such encapsulated small molecule drugs can be slowly released by the diffusion from the polymer layer. In certain embodiments, the encapsulated small molecule drug is a member of the taxane family of anti-tubulin agents. In other embodiments, the encapsulated small molecule drug is paclitaxel. In still other embodiments, the encapsulated small molecule drug is docetaxel.

When the polymers of formula III are used for surface modification and encapsulation of a hydrophobic small molecule drug, the R^{x} groups of the polymer layer may be optionally crosslinked to control the diffusion of the encapsulated drug. Such crosslinking chemistrty is well known in the art and includes such methods described in detail in United States patent application publication number US20060240092, the entirety of which is hereby incorporated herein by reference. Such encapsulated small molecule drugs can be released in a controlled fashion over longer time periods compared to release by diffusion alone. In certain embodiments, the encapsulated small molecule drug is a member of the taxane family of anti-tubulin agents. In other embodiments, the encapsulated small molecule drug is paclitaxel. In still other embodiments, the encapsulated small molecule drug is docetaxel.

Small molecule drugs suitable as R¹, R², and R⁴ groups of the present compounds include, but are not limited to, those having a functional group, or can be modified to include a functional group, suitable for covalently linking to one or more amine or thiol groups incorporated onto the metal substrate. As described herein, such drugs can be linked directly or via a hydrolytically cleavable linker. Such drugs include, without limitation, chemotherapeutic agents or other anti-proliferative agents including alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), angiogenesis inhibitors (Avastin) and hormones (Tamoxifen, Leuprolide, Flutamide, and Megestrol), Gleevec, dexamethasone, and cyclophosphamide. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

In other emboidments, the chemotherapeutic agent is Exemestance (aromasin), Camptosar (irinotecan), Ellence (epirubicin), Femara (Letrozole), Gleevac (imatinib mesylate), Lentaron (formestane), Cytadren/Orimeten (aminoglutethimide), Temodar, Proscar (finasteride), Viadur (leuprolide), Nexavar (Sorafenib), Kytril (Granisetron), Taxotere (Docetaxel), Taxol (paclitaxel), Kytril (Granisetron), Vesanoid (tretinoin) (retin A), XELODA (Capecitabine), Arimidex (Anastrozole), Casodex/Cosudex (Bicalutamide), Faslodex (Fulvestrant), Iressa (Gefitinib), Nolvadex, Istubal, Valodex (tamoxifen citrate), Tomudex (Raltitrexed), Zoladex (goserelin acetate), Leustatin (Cladribine), Velcade (bortezomib), Mylotarg (gemtuzumab ozogamicin), Alimta (pemetrexed), Gemzar (gemcitabine hydrochloride), Rituxan (rituximab), Revlimid (lenalidomide), Thalomid (thalidomide), Alkeran (melphalan), and derivatives thereof.

Other exemplary small molecule drugs include analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolyptics, lipid regulating agents, anti-anginal agents, Cox-2 inhibitors, leukotriene inhibitors, macrolides, muscle relaxants, nutritional agents, opiod analgesics, protease inhibitors, sex hormones, stimulants, muscle relaxants, anti-osteoporosis agents, anti-obesity agents, cognition enhancers, anti-urinary incontinence agents, anti-benign prostate hypertrophy agents, essential fatty acids, non-essential fatty acids, and mixtures thereof.

Other examples of small molecule drugs include treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

In other embodiments, the R¹ group of formula I or the R⁴ group of formula V is a small molecule blood-compatibilizing agent. Such blood-compatibilizing agents are well known in the art and include phosphorylchoine and phosphorylcholine derivatives.

It will also be appreciated that the R² group of formula II can be a small molecule blood-compatibilizing agent attached to the PEG via a hydrolytically stable linkage. In certain embodiments, the R² group is phosphorylchoine or a phosphorylcholine derivative.

Exemplary R¹ groups are set forth in Table 2, below.

TABLE 2
Representative R¹ Groups

| | |
|---|---|
| 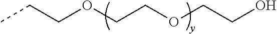 | i |
| 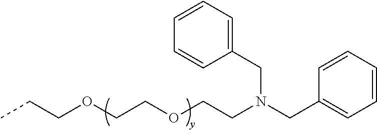 | ii |
| 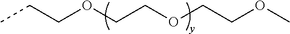 | iii |
| 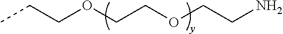 | iv |
| 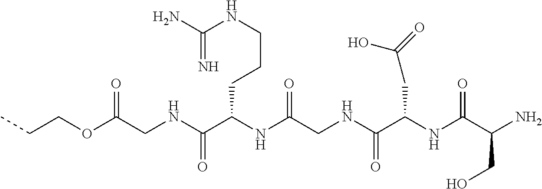 | v |
| 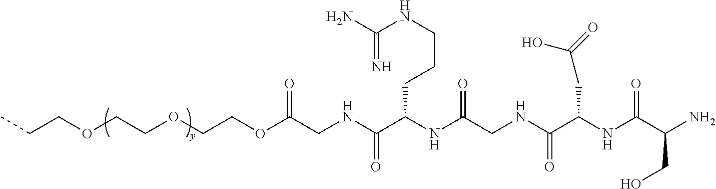 | vi |
| 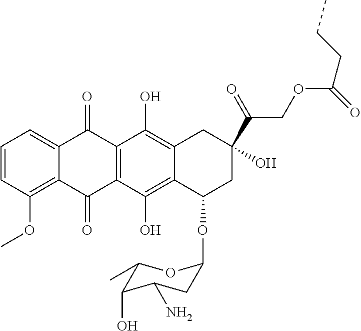 | vii |
| 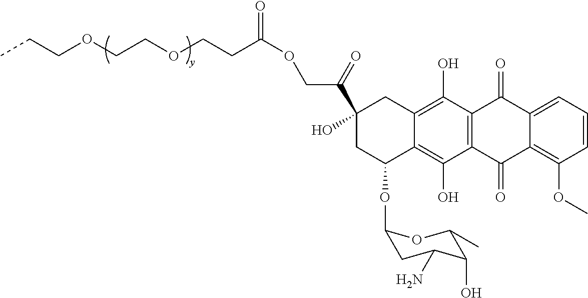 | viii |
| 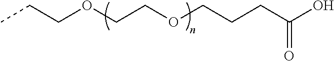 | ix |
| 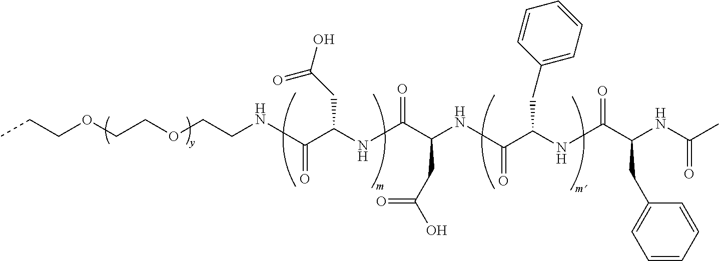 | x |
| 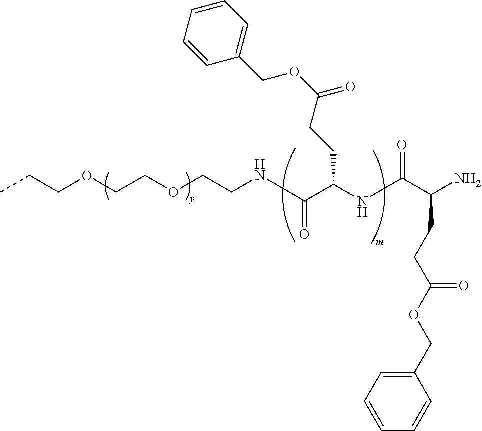 | xi |
| 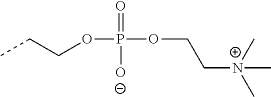 | xii |
| 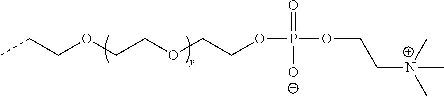 | xiii |
| 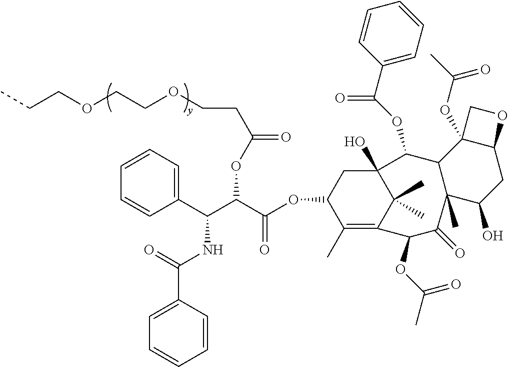 | xiv |
| 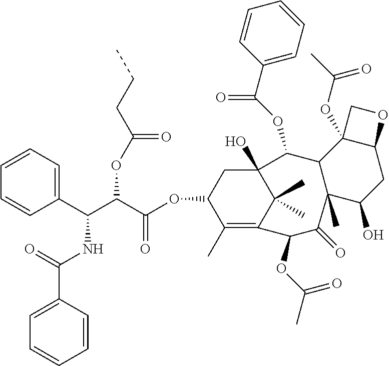 | xv |
| 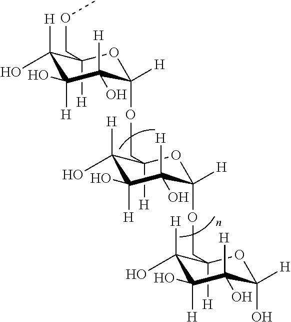 | xvi |
| 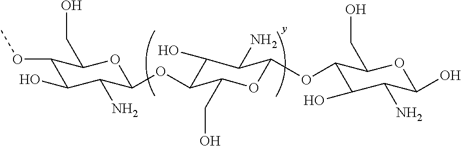 | xvii |
| 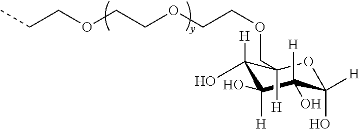 | xviii |
| 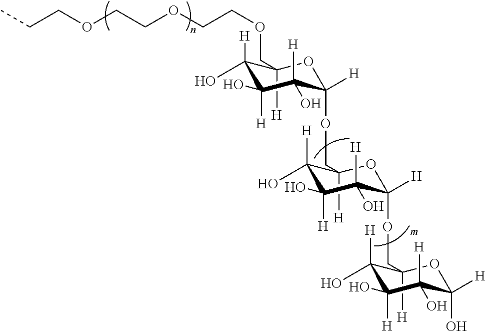 | xix |
| 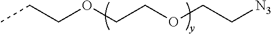 | xx |
| 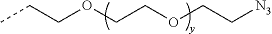 | xxi |
| 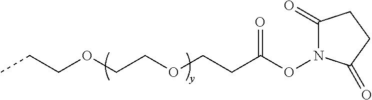 | xxii |
| 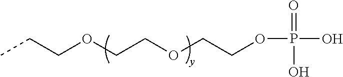 | xxiii |
| 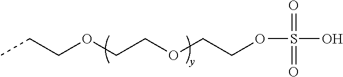 | xxiv |
| 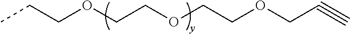 | xxv |
| 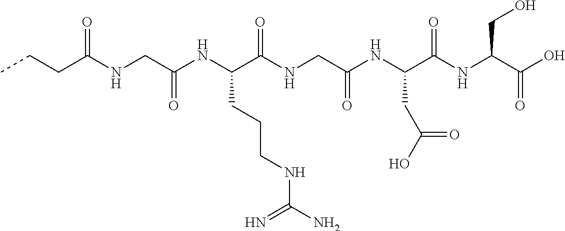 | xxvi |
| 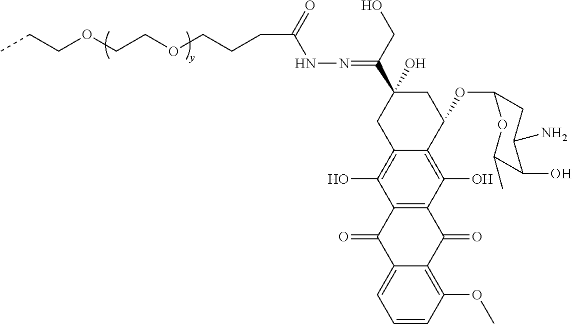 | xxvii |
| 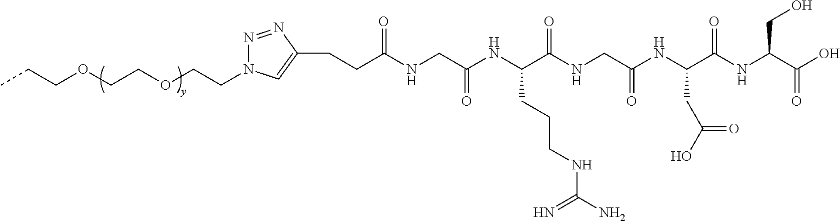 | xxviii |
| 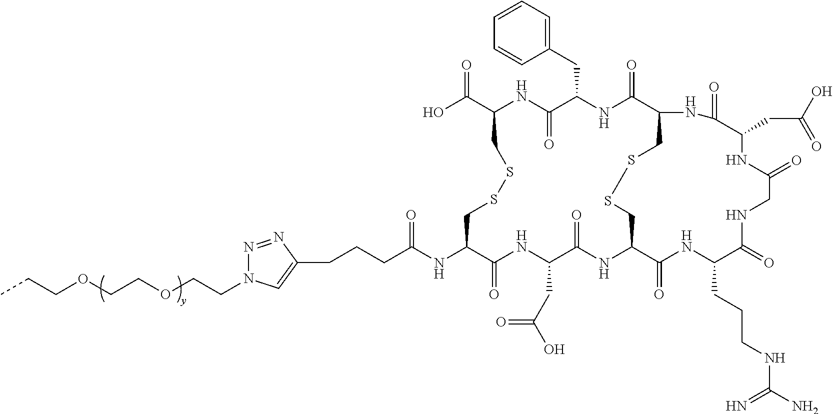 | xxix |
| 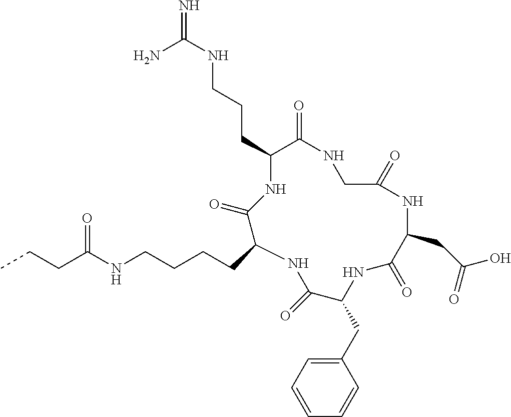 | xxx |
| 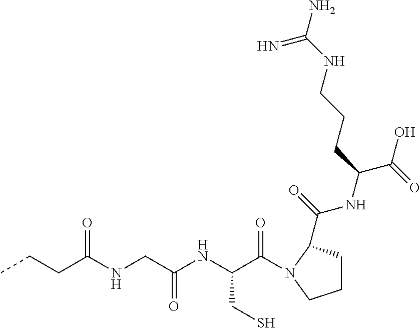 | xxxi |
| 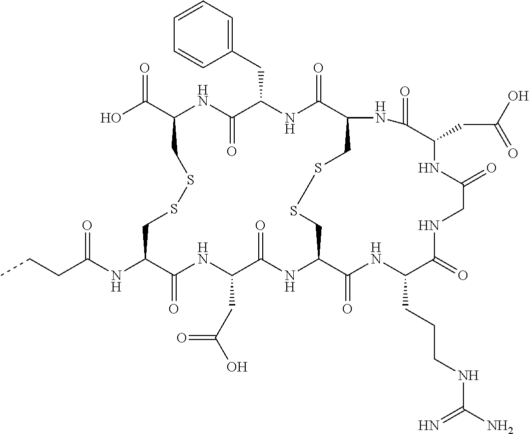 | xxxii |
| 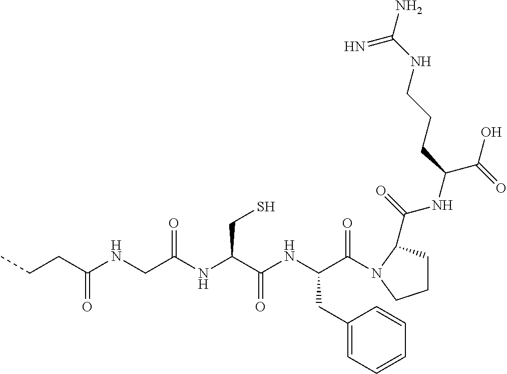 | xxxiii |
| 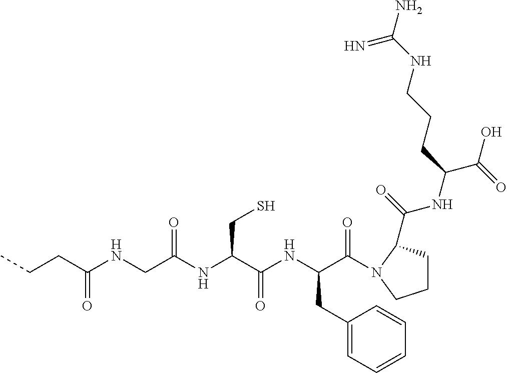 | xxxiv |
| 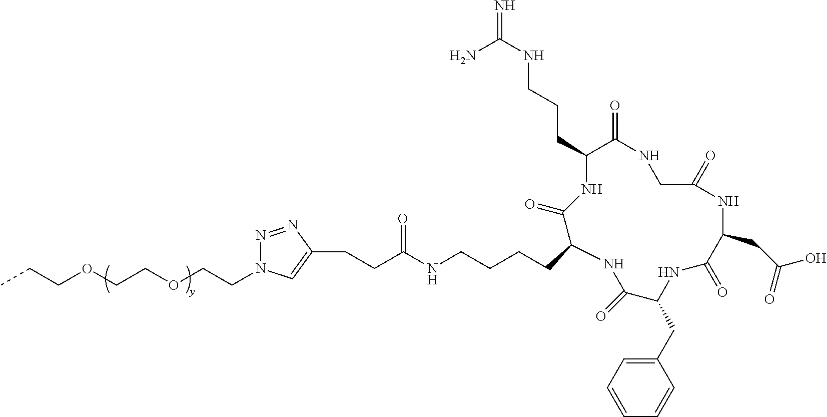 | xxxv |
| 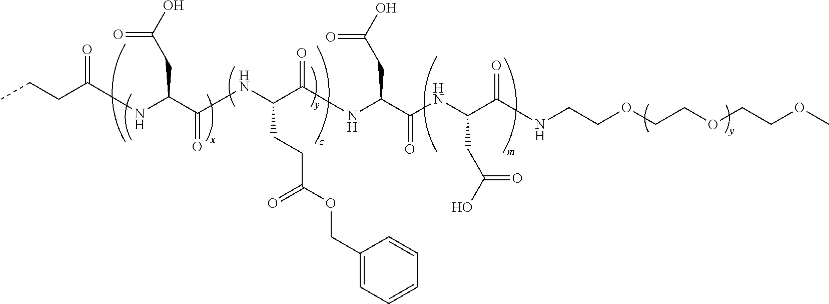 | xxxvi |
| 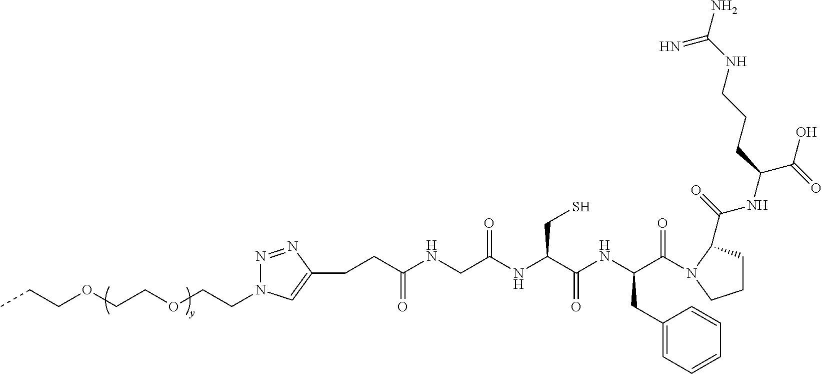 | xxxvii |
| 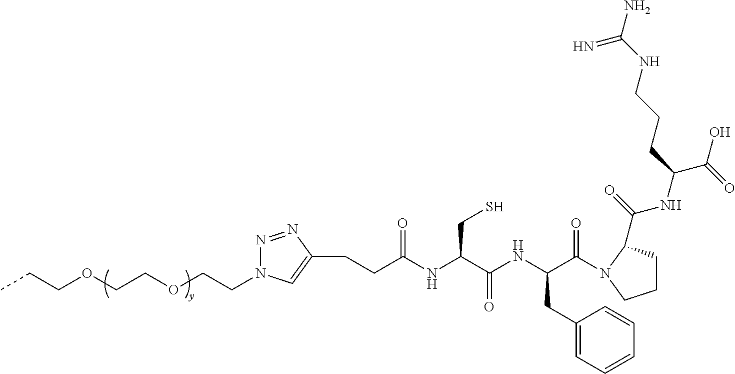 | xxxviii |
| 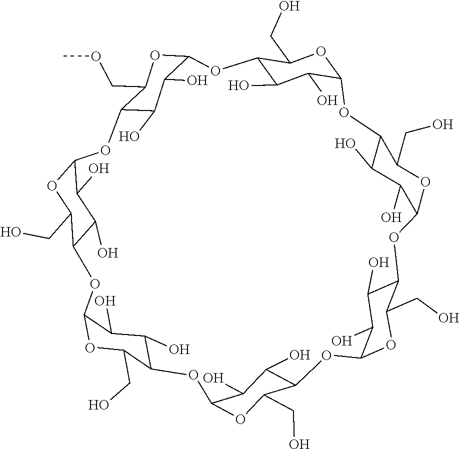 | xxxix |
| 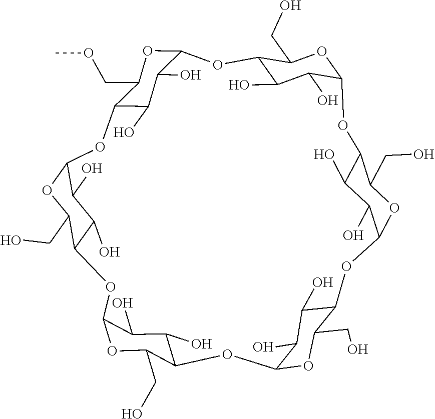 | xxxx |
| 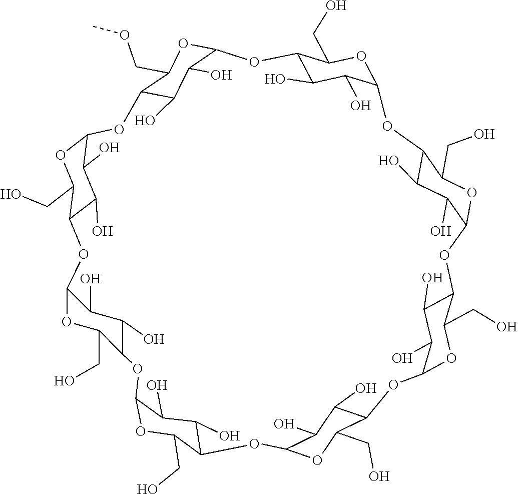 | xxxxi |
| 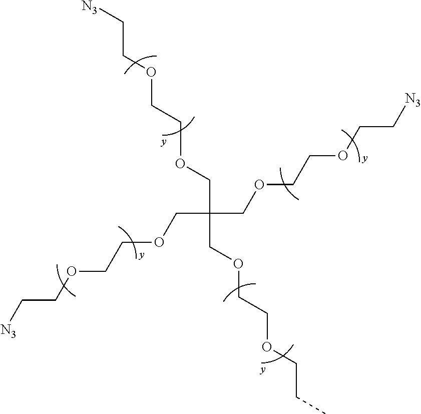 | xxxxii |
| 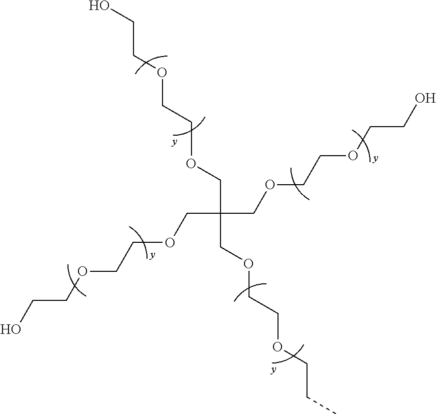 | xxxxiii |
| 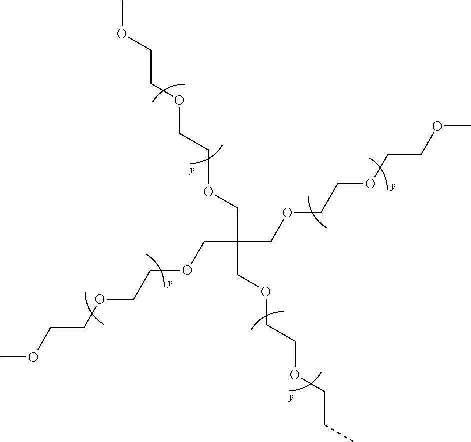 | xxxxiv |
| 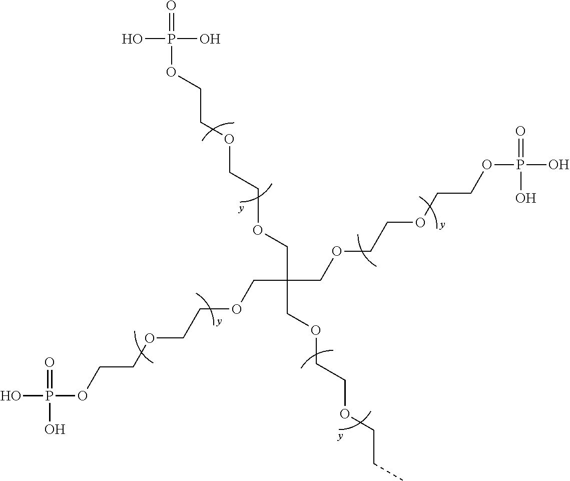 | xxxxv |

wherein each y, m, and m′ is as defined above and described herein.

Additional exemplary R¹ groups are set forth in Table 3, below.

TABLE 3
Representative R¹ Groups

| | |
|---|---|
| 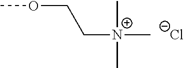 | xii |
| 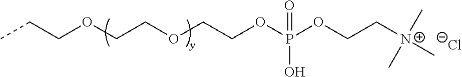 | xiii |
| 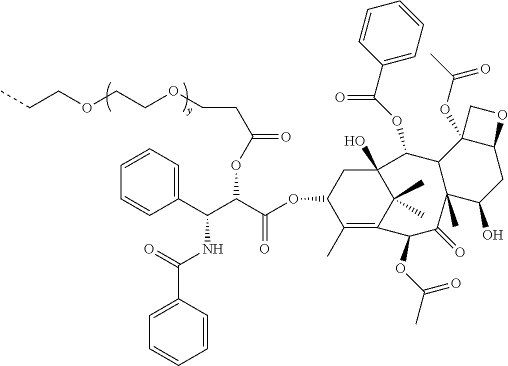 | xiv |
| 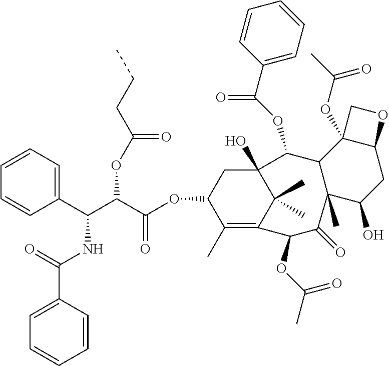 | xv |
| 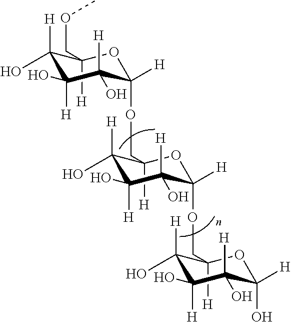 | xvi |
| 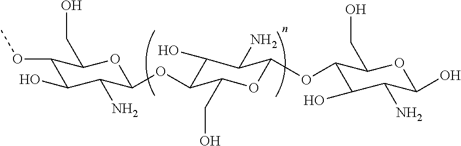 | xvii |
| 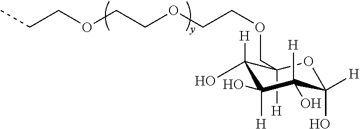 | xviii |
| 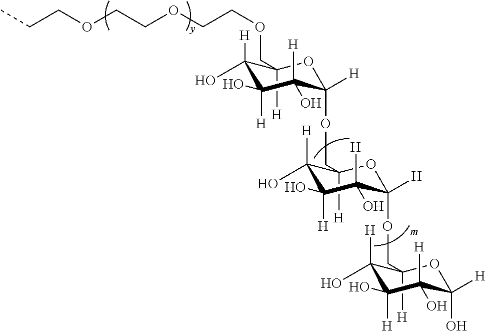 | xix |
| 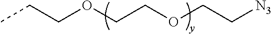 | xx |
| 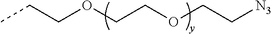 | xxi |
| 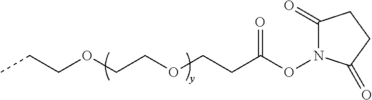 | xxii |

wherein each y is as defined above and described herein.

Exemplary R^{a} groups are set forth in Table 4, below.

TABLE 4
Exemplary R^{a} Groups

| | |
|---|---|
| 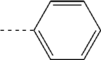 | a |
| - - - -CH₃ | b |
| 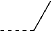 | c |
| 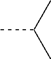 | d |
| 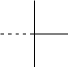 | e |

Exemplary R² groups are set forth in Table 5, below.

TABLE 5
Exemplary R² Groups

| | |
|---|---|
| 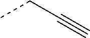 | a |
| 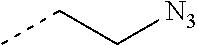 | b |
| 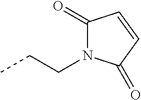 | c |
| 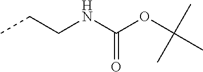 | d |
| 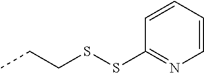 | e |
| 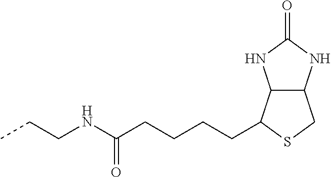 | f |
| 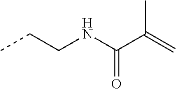 | g |
| 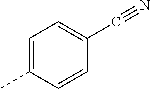 | h |
| 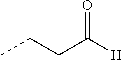 | i |
| 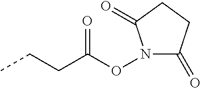 | j |
| 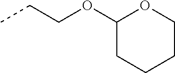 | k |
| 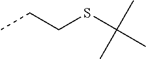 | l |
| - - - -CH₃ | m |

### 4. General Methods for Providing Compounds of the Present Invention

Multiblock copolymers of the present invention are prepared by methods known to one of ordinary skill in the art and those described in detail in United States patent publication number US 20060172914, published Aug. 3, 2006, the entirety of which is hereby incorporated herein by reference. Generally, such multiblock copolymers are prepared by sequentially polymerizing one or more cyclic amino acid monomers onto a hydrophilic polymer having a terminal amine salt wherein said polymerization is initiated by said amine salt. In certain embodiments, said polymerization occurs by ring-opening polymerization of the cyclic amino acid monomers. In other embodiments, the cyclic amino acid monomer is an amino acid NCA, lactam, or imide.

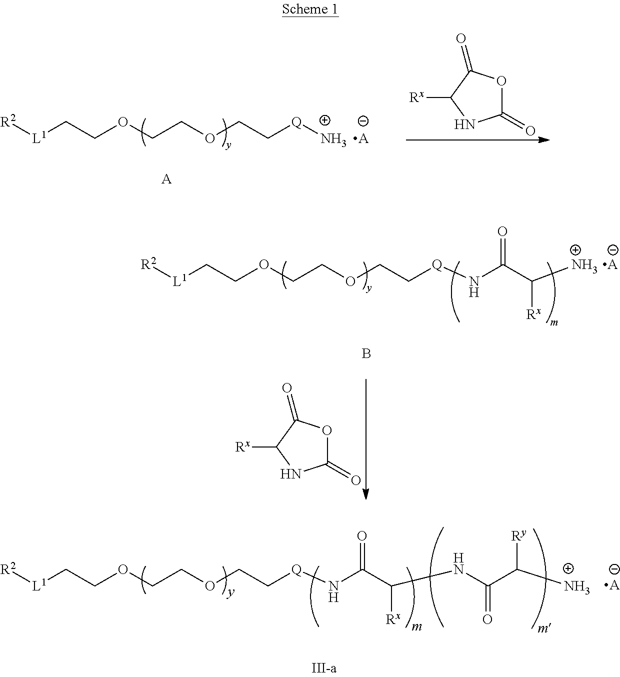

Scheme 1 above depicts a general method for preparing multiblock polymers of the R¹ group of formula I of the present invention. A macroinitiator of formula A is treated with a first amino acid NCA to form a compound of formula B having a first amino acid block. The second amino acid NCA is added to the living polymer of formula B to form a compound of formula III-a having two differing amino acid blocks. Each of the R², L¹, A, y, Q, R^{x}, R^{y}, m, and m′ groups depicted in Scheme 1 are as defined and described in classes and subclasses, singly and in combination, herein.

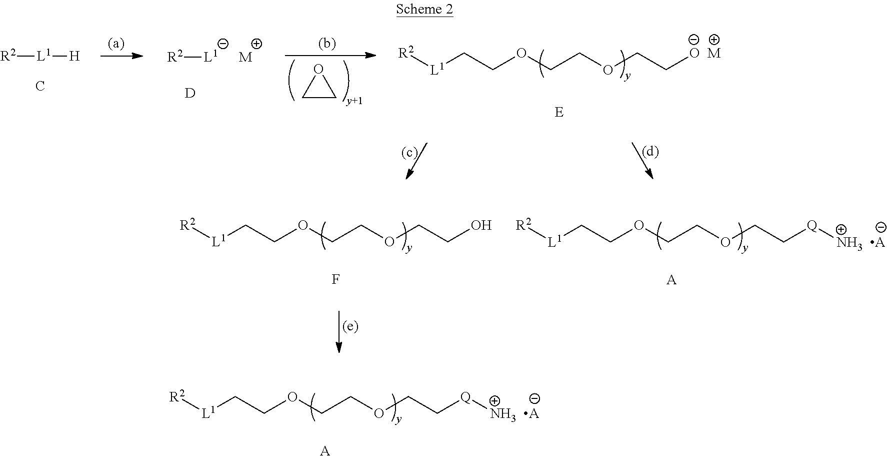

Scheme 2 above shows one exemplary method for preparing the bifunctional PEGs used to prepare the multiblock copolymers of the present invention. As described in United States patent application publication number US20060142506, suitably protected PEG-amines may be formed by terminating the living polymer chain end of a PEG with a terminating agent that contains a suitably protected amine. The suitably protected amine may then be deprotected to generate a PEG that is terminated with a free amine that may subsequently be converted into the corresponding PEG-amine salt macroinitiator. In certain embodiments, the PEG-amine salt macroinitiator of the present invention is prepared directly from a suitably protected PEG-amine by deprotecting said protected amine with an acid. Accordingly, in other embodiments, the terminating agent has suitably protected amino group wherein the protecting group is acid-labile.

Alternatively, suitable synthetic polymers having a terminal amine salt may be prepared from synthetic polymers that contain terminal functional groups that may be converted to amine salts by known synthetic routes. In certain embodiments, the conversion of the terminal functional groups to the amine salts is conducted in a single synthetic step. In other embodiments, the conversion of the terminal functional groups to the amine salts is achieved by way of a multi-step sequence. Functional group transformations that afford amines, amine salts, or protected amines are well known in the art and include those described in Larock, R. C., “Comprehensive Organic Transformations,” John Wiley & Sons, New York, 1999.

At step (a), the polymerization initiator is treated with a suitable base to form D. A variety of bases are suitable for the reaction at step (a). Such bases include, but are not limited to, potassium naphthalenide, diphenylmethyl potassium, triphenylmethyl potassium, and potassium hydride. At step (b), the resulting anion is treated with ethylene oxide to form the polymer E. Polymer E can be transformed at step (d) to a compound of formula A directly by terminating the living polymer chain-end of E with a suitable polymerization terminator to afford a compound of formula A. Alternatively, polymer E may be quenched at step (c) to form the hydroxyl compound F. Compound F is then derivatized to afford a compound of formula A by methods known in the art, including those described herein. Each of the R², L¹, A, y, and Q groups depicted in Scheme 2 are as defined and described in classes and subclasses, singly and in combination, herein.

Although certain exemplary embodiments are depicted and described above and herein, it will be appreciated that compounds of the invention can be prepared according to the methods described generally above using appropriate starting materials by methods generally available to one of ordinary skill in the art. Additional embodiments are exemplified in more detail herein.

### 5. Uses

The modified metal surfaces in accordance with the present invention are useful for a multitude of uses. In certain embodiments, the covalently modified metal surfaces are useful in any application where metal is typically coated in a non-covalent fashion. Such applications include coated implantable medical devices. Such implantable devices include prostheses, artificial valves, vascular grafts, stents, catheters, and the like. These, and other such devices, are described in more detail below. In certain embodiments, the implantable device is a cardiovascular device, a neurosurgical device, a gastrointestinal device, a genitourinary device, a phthalmologic implant, an otolaryngology device, a plastic surgery implant, or an orthopedic implant.

Certain disorders are associated with the tissue trauma resulting from a medical procedure, such as angioplasty, or from the implantation of a medical device. For example, restenosis is a re-narrowing or blockage of an artery at the same site where treatment, such as an angioplasty or stent procedure, has already taken place. One cause of restenosis is tissue growth at the site of treatment characterized by a proliferation of the smooth muscle cells that normally line blood vessels.

Recent developments in the ongoing battle to reduce restenosis include the drug-eluting stent which has a medication coated on it to reduce the proliferation of cells that can cause restenosis. There is a continuing need to develop stents and other implantable devices coated with anti-proliferative agents advantageous for treating or preventing disorders associated with tissue trauma caused by implantable devices.

According to another aspect, the present invention provides a method for treating or preventing disorders associated with tissue trauma caused by implantable devices wherein said method comprises providing an implantable device, wherein at least a portion of said implantable device is a covalently modified metal surface in accordance with the present invention, and implanting said device in a patient. Said method is useful for treating or preventing, for example, restenosis of blood vessels subject to traumas such as angioplasty and stenting.

In other embodiments, the present invention provides an implantable device, wherein at least a portion of said device is a covalently modified metal surface in accordance with the present invention. In still other embodiments, the present invention provides a PEGylated implantable device, wherein at least a portion of said device is a metal surface comprising PEG covalently bonded thereto via amine or thiol groups incorporated thereon.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a composition comprising an anti-proliferative compound. Such coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. However, none of these coatings is covalently bonded to the device.

As described generally herein, one method of treating restenosis is to coat an implantable device with an anti-proliferative compound. Although the present invention contemplates covalently modified metal surfaces which incorporate small molecule drugs, one of ordinary skill in the art will appreciate that the present invention also contemplates covalently modified metal surfaces which do not incorporate small molecule drugs. Without wishing to be bound by any particular theory, it is believed that the “stealth” properties of PEG are particularly suited for implantable devices having at least a portion which is a metal surface covalently modified with PEG or a functionalized PEG. It is believed that the PEG itself will not induce restenosis, or other tissue injury caused by an implantable device, thereby negating the need for incorporation of an anti-proliferative or other small molecule drug. Thus, the present invention also provides an implantable device covalently modified in accordance with the present invention. In certain embodiments, the implantable device is covalently modified by a functionalized PEG. In other embodiments, the implantable device is covalently modified by a functionalized PEG of formula II as defined above and in classes and subclasses described herein.

As discussed above, the present devices are useful for treating or preventing restenosis of blood vessels subject to traumas such as angioplasty and stenting. For example, it is contemplated that such implanted medical devices include as tubings, shunts, catheters, artificial implants, pins, electrical implants such as pacemakers, and especially for arterial or venous stents, including balloon-expandable stents.

In certain embodiments, methods of the present invention are used for coating stents, or a metallic substrate to be made into a stent. A stent is typically an open tubular structure that has a pattern (or patterns) of apertures extending from the outer surface of the stent to the lumen. It is commonplace to make stents of biocompatible metallic materials, with the patterns cut on the surface with a laser machine. The stent can be electro-polished to minimize surface irregularities since these irregularities can trigger an adverse biological response. However, stents may still stimulate foreign body reactions that result in thrombosis or restenosis. To avoid these complications, a variety of stent coatings and compositions have been proposed to reduce the incidence of these complications or other complications and restore tissue function by itself or by delivering therapeutic compound to the lumen. For example, compounds having antiproliferative and anti-inflammatory activities have been evaluated as stent coatings, and have shown promise in preventing restenosis (See, for example, Presbitero P. et al., “Drug eluting stents do they make the difference?”, Minerva Cardioangiol, 2002, 50(5):431-442; Ruygrok P. N. et al., “Rapamycin in cardiovascular medicine”, Intern. Med. J., 2003, 33(3):103-109; and Marx S. O. et al., “Bench to bedside: the development of rapamycin and its application to stent restenosis”, Circulation, 2001, 104(8):852-855).

In certain embodiments, the present invention provides a stent, having at least a portion which is a covalently modified metal surface, for insertion into an artery or vein following balloon angioplasty. According to one aspect, the present invention provides a method of inhibiting arterial restenosis or arterial occlusion following vascular trauma comprising insertion into a subject in need thereof, a stent, having at least a portion which is a covalently modified metal surface. In the practice of the method, the subject may be a coronary bypass, vascular surgery, organ transplant or coronary or any other arterial angioplasty patient, for example.

In another aspect, the invention encompasses implants and surgical or medical devices, including stents and grafts, having at least a portion which is a covalently modified metal surface. In certain embodiments, the devices include compounds which inhibit smooth muscle cell proliferation. Representative examples of implants and surgical or medical devices contemplated by the present invention include cardiovascular devices (e.g., implantable venous catheters, venous ports, tunneled venous catheters, chronic infusion lines or ports, including hepatic artery infusion catheters, pacemaker wires, implantable defibrillators); neurologic/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, implantable meshes for hernias, suspensions or solid implants to prevent surgical adhesions, including meshes); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy); otolaryngology devices (e.g., ossicular implants, Eustachian tube splints or stents for glue ear or chronic otitis as an alternative to transtempanic drains); and orthopedic implants (e.g., cemented orthopedic prostheses).

In other embodiments of the invention, the implant or device contemplated by the present invention provides a uniform, predictable, prolonged release of the therapeutic agent, or composition thereof, into the tissue surrounding the implant or device once it has been deployed. For vascular stents, in addition to the above properties, the composition should not render the stent thrombogenic (causing blood clots to form), or cause significant turbulence in blood flow (more than the stent itself would be expected to cause if it was uncoated). In certain embodiments, said therapeutic agent is an anti-proliferative compound. In still other embodiments, said therapeutic agent is Paclitaxel.

In the case of stents, a wide variety of stents may be covalently modified in accordance with the present invention including esophageal stents, gastrointestinal stents, vascular stents, biliary stents, colonic stents, pancreatic stents, ureteric and urethral stents, lacrimal stents, Eustachian tube stents, fallopian tube stents and tracheal/bronchial stents (See, for example, U.S. Pat. No. 6,515,016, the entire contents of which are incorporated herein by reference). Stents may be readily obtained from commercial sources, or constructed in accordance with well-known techniques. Representative examples of stents include those described in U.S. Pat. No. 4,768,523, entitled “Hydrogel Adhesive”; U.S. Pat. No. 4,776,337, entitled “Expandable Intraluminal Graft, and Method and Apparatus for Implanting and Expandable Intraluminal Graft”; U.S. Pat. No. 5,041,126 entitled “Endovascular Stent and Delivery System”; U.S. Pat. No. 5,052,998 entitled “Indwelling Stent and Method of Use”; U.S. Pat. No. 5,064,435 entitled “Self-Expanding Prosthesis Having Stable Axial Length”; U.S. Pat. No. 5,147,370, entitled “Nitinol Stent for Hollow Body Conduits”; U.S. Pat. No. 5,176,626, entitled “Indwelling Stent”; U.S. Pat. No. 6,344,028 entitled “Replenishable Stent and Delivery System”; and U.S. Pat. No. 5,328,471, entitled “Method and Apparatus for Treatment of Focal Disease in Hollow Tubular Organs and Other Tissue Lumens.”

As discussed above, the stent covalently modified in accordance with the present invention may be used to eliminate a vascular obstruction and prevent restenosis or reduce the rate of restenosis. Within other aspects of the present invention, such stents are provided for expanding the lumen of a body passageway. Specifically, a stent having a generally tubular structure, and at least a portion which is covalently modified metal, may be inserted into the passageway, such that the passageway is expanded. In certain embodiments, the stent may be used to eliminate a biliary, gastrointestinal, esophageal, tracheal/bronchial, urethral or vascular obstruction.

In other embodiments, methods are provided for preventing restenosis, comprising inserting a stent into an obstructed blood vessel, the stent having a generally tubular structure, at least a portion of the surface of the structure being covalently modified in accordance with the present invention, such that the obstruction is eliminated and smooth muscle cell proliferation is prevented or inhibited.

Within other aspects of the present invention, methods are provided for expanding the lumen of a body passageway, comprising inserting a stent into the passageway, the stent having a generally tubular structure, at least a portion of the surface of the structure being covalently modified in accordance with the present invention, such that the passageway is expanded. In certain embodiments, the lumen of a body passageway is expanded in order to eliminate a biliary, gastrointestinal, esophageal, tracheal/bronchial, urethral and/or vascular obstruction.

In certain embodiments, methods are provided for eliminating biliary obstructions, comprising inserting a biliary stent into a biliary passageway, the stent having a generally tubular structure, at least a portion of the surface of the structure being covalently modified in accordance with the present invention, such that the biliary obstruction is eliminated. For example, tumor overgrowth of the common bile duct results in progressive cholestatic jaundice which is incompatible with life. Generally, the biliary system which drains bile from the liver into the duodenum is most often obstructed by (1) a tumor composed of bile duct cells (cholangiocarcinoma), (2) a tumor which invades the bile duct (e.g., pancreatic carcinoma), or (3) a tumor which exerts extrinsic pressure and compresses the bile duct (e.g., enlarged lymph nodes). Both primary biliary tumors, as well as other tumors which cause compression of the biliary tree may be treated utilizing stents, implants and other surgical or medical devices, at least a portion of which is metal which is covalently modified in accordance with the present invention.

One example of primary biliary tumors are adenocarcinomas (which are also called Klatskin tumors when found at the bifurcation of the common hepatic duct). These tumors are also referred to as biliary carcinomas, choledocholangiocarcinomas, or adenocarcinomas of the biliary system. Benign tumors which affect the bile duct (e.g., adenoma of the biliary system), and, in rare cases, squamous cell carcinomas of the bile duct and adenocarcinomas of the gallbladder, may also cause compression of the biliary tree and therefore, result in biliary obstruction. Compression of the biliary tree is most commonly due to tumors of the liver and pancreas which compress and therefore obstruct the ducts. Most of the tumors from the pancreas arise from cells of the pancreatic ducts. This is a highly fatal form of cancer (5% of all cancer deaths; 26,000 new cases per year in the U.S.) with an average of 6 months survival and a 1 year survival rate of only 10%. When these tumors are located in the head of the pancreas they frequently cause biliary obstruction, and this detracts significantly from the quality of life of the patient. While all types of pancreatic tumors are generally referred to as “carcinoma of the pancreas” there are histologic subtypes including: adenocarcinoma, adenosquamous carcinoma, cystadenocarcinoma, and acinar cell carcinoma. Hepatic tumors, as discussed above, may also cause compression of the biliary tree, and therefore cause obstruction of the biliary ducts.

A biliary stent is first inserted into a biliary passageway in one of several ways: from the top end by inserting a needle through the abdominal wall and through the liver (a percutaneous transhepatic cholangiogram or “PTC”); from the bottom end by cannulating the bile duct through an endoscope inserted through the mouth, stomach, and duodenum (an endoscopic retrograde cholangiogram or “ERCP”); or by direct incision during a surgical procedure. A preinsertion examination, PTC, ERCP, or direct visualization at the time of surgery is optionally performed to determine the appropriate position for stent insertion. A guidewire is then advanced through the lesion, and over this a delivery catheter is passed to allow the stent to be inserted in its collapsed form. If the diagnostic exam was a PTC, the guidewire and delivery catheter is inserted via the abdominal wall, while if the original exam was an ERCP the stent may be placed via the mouth. The stent is then positioned under radiologic, endoscopic, or direct visual control taking particular care to place it precisely across the narrowing in the bile duct. The delivery catheter is then removed leaving the stent standing as a scaffolding which holds the bile duct open. A further cholangiogram may be performed to document that the stent is appropriately positioned.

In certain embodiments, methods are provided for eliminating esophageal obstructions, comprising inserting an esophageal stent into an esophagus, the stent having a generally tubular structure, at least a portion of the surface of the structure being covalently modified in accordance with the present invention, such that the esophageal obstruction is eliminated. For example, the esophagus is the hollow tube which transports food and liquids from the mouth to the stomach. Cancer of the esophagus or invasion by cancer arising in adjacent organs (e.g., cancer of the stomach or lung) results in the inability to swallow food or saliva. In certain embodiments, a pre-insertion examination, usually a barium swallow or endoscopy is performed in order to determine the appropriate position for stent insertion. A catheter or endoscope may then be positioned through the mouth, and a guidewire is advanced through the blockage. A stent delivery catheter is passed over the guidewire under radiologic or endoscopic control, and a stent is placed precisely across the narrowing in the esophagus. A post-insertion examination, usually a barium swallow x-ray, may be utilized to confirm appropriate positioning.

In certain embodiments, methods are provided for eliminating colonic obstructions, comprising inserting a colonic stent into a colon, the stent having a generally tubular structure, at least a portion of the surface of the structure being covalently modified in accordance with the present invention, such that the colonic obstruction is eliminated. For example, the colon is the hollow tube which transports digested food and waste materials from the small intestines to the anus. Cancer of the rectum and/or colon or invasion by cancer arising in adjacent organs (e.g., cancer of the uterus, ovary, bladder) results in the inability to eliminate feces from the bowel. In certain embodiments, a pre-insertion examination, usually a barium enema or colonoscopy is performed in order to determine the appropriate position for stent insertion. A catheter or endoscope may then be positioned through the anus, and a guidewire is advanced through the blockage. A stent delivery catheter is passed over the guidewire under radiologic or endoscopic control, and a stent is placed precisely across the narrowing in the colon or rectum. A post-insertion examination, usually a barium enema x-ray, may be utilized to confirm appropriate positioning.

In certain embodiments, methods are provided for eliminating tracheal/bronchial obstructions, comprising inserting a tracheal/bronchial stent into a trachea or bronchi, the stent having a generally tubular structure, at least a portion of the surface of the structure being covalently modified in accordance with the present invention, such that the tracheal/bronchial obstruction is eliminated. For example, the trachea and bronchi are tubes which carry air from the mouth and nose to the lungs. Blockage of the trachea by cancer, invasion by cancer arising in adjacent organs (e.g., cancer of the lung), or collapse of the trachea or bronchi due to chondromalacia (weakening of the cartilage rings) results in inability to breathe. In certain embodiments, a pre-insertion examination, usually an endoscopy, is performed in order to determine the appropriate position for stent insertion. A catheter or endoscope is then positioned through the mouth, and a guidewire advanced through the blockage. A delivery catheter is then passed over the guidewire in order to allow a collapsed stent to be inserted. The stent is placed under radiologic or endoscopic control in order to place it precisely across the narrowing. The delivery catheter may then be removed leaving the stent standing as a scaffold on its own. A post-insertion examination, usually a bronchoscopy may be utilized to confirm appropriate positioning.

In certain embodiments, methods are provided for eliminating urethral obstructions, comprising inserting a urethral stent into a urethra, the stent having a generally tubular structure, at least a portion of the surface of the structure being covalently modified in accordance with the present invention, such that the urethral obstruction is eliminated. For example, the urethra is the tube which drains the bladder through the penis. Extrinsic narrowing of the urethra as it passes through the prostate, due to hypertrophy of the prostate, occurs in virtually every man over the age of 60 and causes progressive difficulty with urination. In certain embodiments, a pre-insertion examination, usually an endoscopy or urethrogram is first performed in order to determine the appropriate position for stent insertion, which is above the external urinary sphincter at the lower end, and close to flush with the bladder neck at the upper end. An endoscope or catheter is then positioned through the penile opening and a guidewire advanced into the bladder. A delivery catheter is then passed over the guidewire in order to allow stent insertion. The delivery catheter is then removed, and the stent expanded into place. A post-insertion examination, usually endoscopy or retrograde urethrogram, may be utilized to confirm appropriate position.

In certain embodiments, methods are provided for eliminating vascular obstructions, comprising inserting a vascular stent into a blood vessel, the stent having a generally tubular structure, at least a portion of the surface of the structure being covalently modified in accordance with the present invention, such that the vascular obstruction is eliminated. For example, stents may be placed in a wide array of blood vessels, both arteries and veins, to prevent recurrent stenosis at the site of failed angioplasties, to treat narrowings that would likely fail if treated with angioplasty, and to treat post-surgical narrowings (e.g., dialysis graft stenosis). Suitable sites include, but are not limited to, the iliac, renal, and coronary arteries, the superior vena cava, and in dialysis grafts. In certain embodiments, angiography is first performed in order to localize the site for placement of the stent. This is typically accomplished by injecting radiopaque contrast through a catheter inserted into an artery or vein as an x-ray is taken. A catheter may then be inserted either percutaneously or by surgery into the femoral artery, brachial artery, femoral vein, or brachial vein, and advanced into the appropriate blood vessel by steering it through the vascular system under fluoroscopic guidance. A stent may then be positioned across the vascular stenosis. A post-insertion angiogram may also be utilized in order to confirm appropriate positioning.

Compositions comprising one or more therapeutic agents can be coated on a device according to the present invention, which is then implanted to provide localized delivery of the therapeutic agent or agents contained therein. In certain embodiments, said therapeutic agent is an anti-proliferative compound. In still other embodiments, said therapeutic agent is Paclitaxel. General methods for delivering the therapeutic agent or agents contained within the coating on said device to targeted areas of the body have been described, for example, in U.S. Pat. No. 5,651,986. Such localized delivery is useful for, among other things, inhibiting the growth of a tumor. This method avoids the systemic levels of the chemotherapeutic agent or agents often associated with toxicity. The localized delivery of the therapeutic agent is achieved by implanting a device, coated with a composition of the present invention, proximally to the tumor. The therapeutic agent is typically released from the device by diffusion, degradation of the matrix, or a combination thereof. Thus, another aspect of the present invention relates to a method for inhibiting growth of a tumor, in a patient in need thereof, comprising implanting a device, coated with a composition as described herein, for administering localized delivery of the therapeutic agent.

As described above and herein, one aspect of the present invention provides an implantable device, wherein at least a portion of said device is a covalently modified metal surface in accordance with the present invention. In certain aspects of the invention, a provided implantable device is covalently modified with a moiety that is suitable for cell growth. Thus, in certain embodiments, a provided implantable device can function as a scaffold for cells to grow upon thereby affecting integration of the device with surrounding tissue. By way of example, it is contemplated that an orthopedic implant is covalently modified with a compound of formula I in accordance with the present invention whereby the R¹ group comprises a recognition group suitable for bone growth. Such recognition groups are well known in the art. In certain embodiments, the orthopedic implant is a joint implant, such as hip or knee.

### EXAMPLES

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

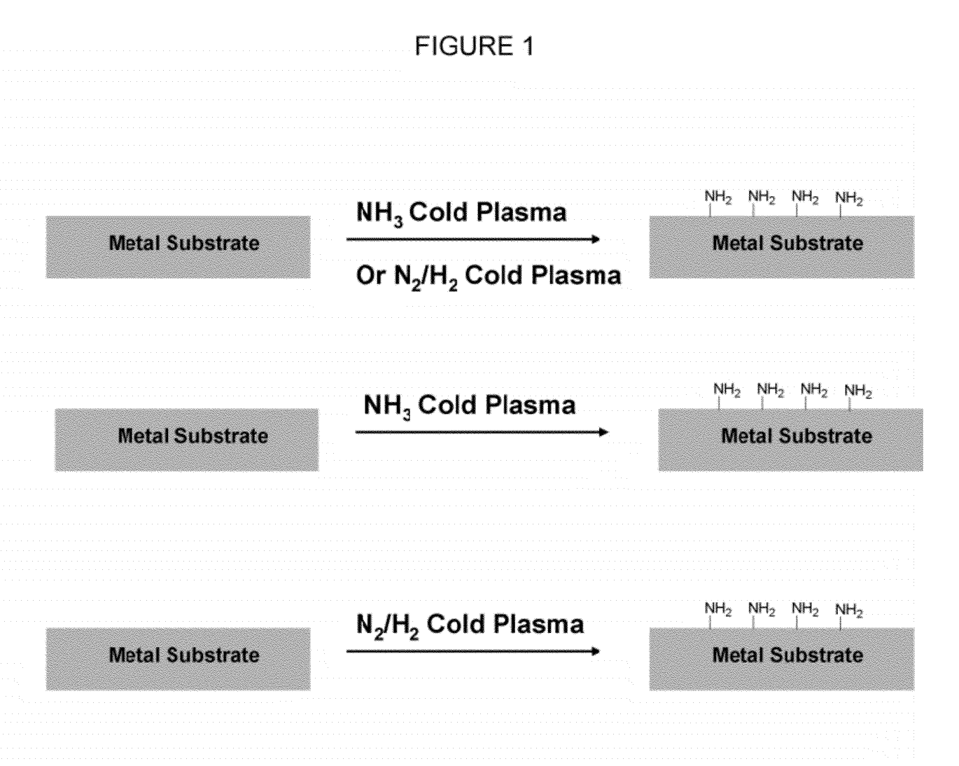

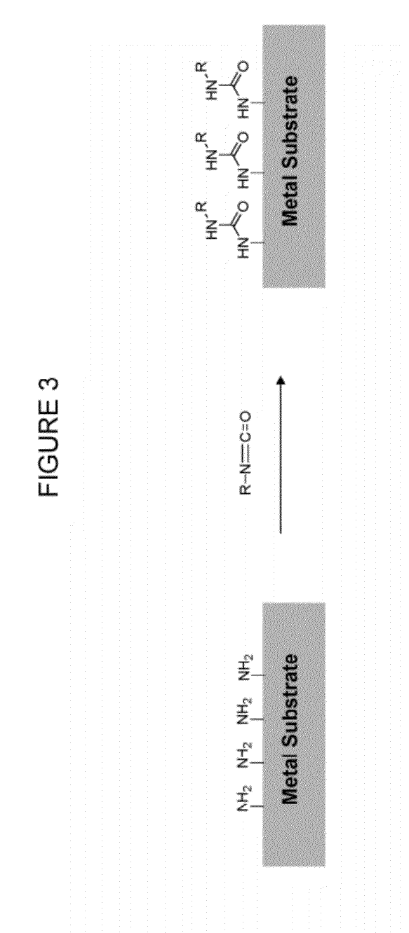

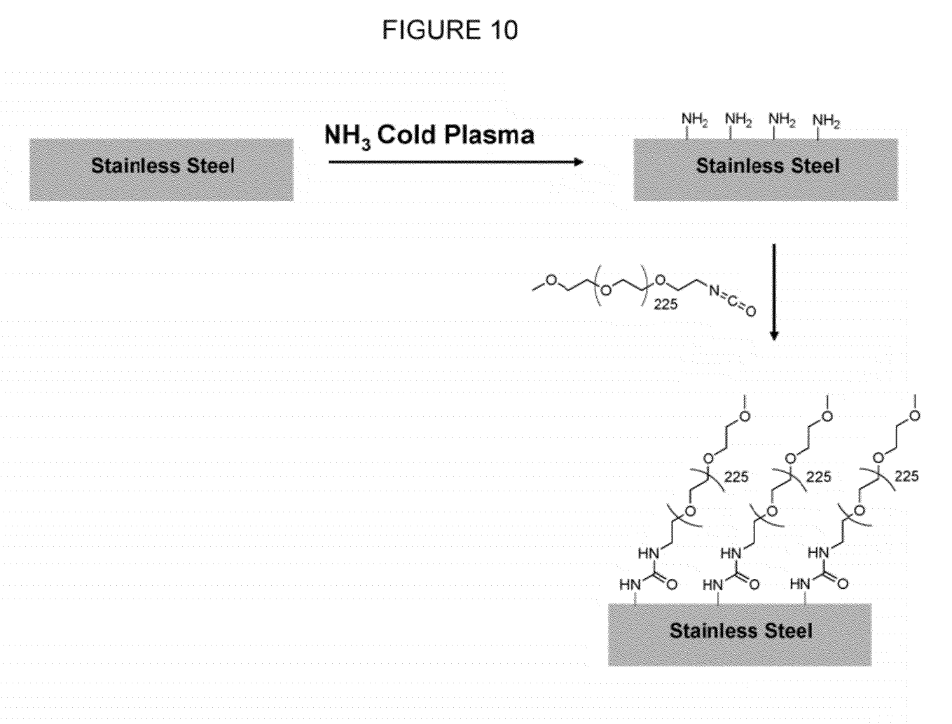

Examples of methods for covalent modification of metal surfaces are depicted in FIGS. 1-11.

### Preparation of Bifunctional PEGs and Multiblock Copolymers of the Present Invention

As described generally above, multiblock copolymers of the present invention are prepared using the heterobifunctional PEGs described herein and in United States patent application publication number US20060142506, the entirety of which is hereby incorporated herein by reference. The preparation of multiblock polymers in accordance with the present invention is accomplished by methods known in the art, including those described in detail in United States patent application publication number US20060172914, the entirety of which is hereby incorporated herein by reference.

### Example 1

### Ammonia Plasma Treatment

A ½″×½″×⅛″ 316 L stainless steel coupon was cleaned with an argon plasma process (250 mtorr Ar₂, 100° C., 4000 watts for 15 minutes at a flow of 2.5 slm). The freshly cleaned coupon was then exposed to an ammonia plasma treatment (250 mtorr NH₃, 100° C., 4000 watts for 15 minutes flow of 2.5 slm). Contact angle was found to be 8°. See FIG. 1.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A method for covalently modifying a metal surface, comprising the steps of introducing amine or thiol groups onto a metal substrate and covalently bonding a polymer or small molecule organic moiety onto the resulting hydrophilic metal surface.

2. The method according to claim 1, wherein the metal substrate comprises iron.

3. The method according to claim 1, wherein the metal substrate is selected from a stainless steel, a cobalt alloy, a titanium alloy, an iron alloy, steel, stainless steel, austenitic stainless steel, Type 316 stainless steel, ferritic stainless steel, martensitic stainless steel, duplex stainless steel, cobalt, a cobalt alloy, a cobalt-chromium alloy, stellite alloys, Vitallium®, titanium, titanium alloy, or a super-alloy.

4. A method for preparing a covalently modified metal surface, comprising the steps of:
(a) modifying a metal substrate to incorporate thereon a plurality of amine or thiol groups;
(b) providing a compound of formula I:
R¹—W I
wherein:
wherein:
R¹ is a natural or synthetic polymer or copolymer group or a small molecule organic group;
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
and
(c) coupling the compound of formula I to one or more of the amine or thiol groups on the metal surface.

5. The method according to claim 4, wherein step (c) is performed by condensation or dehydration reaction.

6. The method according to claim 4, wherein R¹ is poly(ethylene glycol) (PEG), a heterobifunctional PEG, a branched PEG, PEG-b-PAA-b-PAA block copolymers, PEG-b-PAA-b-PEG block copolymers, PEG-b-polyester-b-PEG block copolymers, PEG-b-PAA block copolymers, dextran, chitosan, amylose, amylopectin, glycogen, xanthan, gellan, or pullulun.

7. The method according to claim 4, wherein R¹ is a monosaccharide, a disaccharide, a phosphorylcholine, a phosoplipid, a cyclodextran, or a small molecule drug.

8. A method for preparing a covalently modified metal surface, comprising the steps of:
(a) providing a metal surface having a plurality of amine or thiol groups;
(b) providing a compound of formula I:
R¹—W I
wherein:
R¹ is a natural or synthetic polymer or copolymer group or a small molecule organic group;
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
and
(c) coupling the compound of formula I to one or more of the amine or thiol groups on the metal surface.

9. The method according to claim 8, wherein R¹ is a group of formula II: 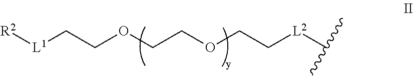
or a salt thereof, wherein:
y is 0-2500;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

10. The method according to claim 9, wherein R¹ is a group of formula II-d:

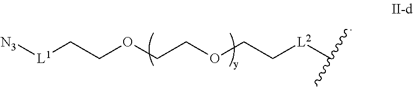

11. The method according to claim 9, further comprising the step of coupling the azide-terminal end to a suitable group via Click chemistry.

12. The method according to claim 8, wherein R¹ is a block copolymer group of formula III: 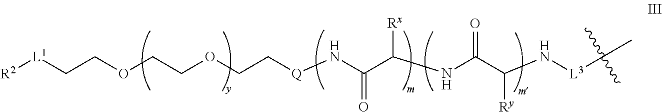
wherein:
y is 1-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L³ is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

13. The method according to claim 8, wherein R¹ is a block copolymer group of formula IV: 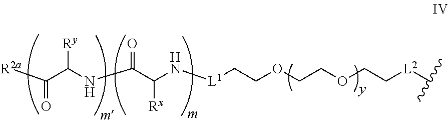
wherein:
y is 10-2500;
m is 1 to 1000;
m′ is 0 to 1000;
R^{x} and R^{y} are each independently a natural or unnatural amino acid side-chain group, wherein R^{x} and R^{y} are different from each other;
R² is hydrogen, halogen, NO₂, CN, N₃, —N═C═O, —C(R)═NN(R)₂, —P(O)(OR)₂, —P(O)(X′)₂, a 9-30 membered crown ether, a mono-protected amine, a di-protected amine, a protected aldehyde, a protected hydroxyl, a protected carboxylic acid, a protected thiol, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X′ is independently halogen;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
Q is a valence bond or a bivalent, saturated or unsaturated, straight or branched C₁₋₁₂ alkylene chain, wherein 0-6 methylene units of Q are independently replaced by -Cy-, —O—, —NH—, —S—, —OC(O)—, —C(O)O—, —C(O)—, —SO—, —SO₂—, —NHSO₂—, —SO₂NH—, —NHC(O)—, —C(O)NH—, —OC(O)NH—, or —NHC(O)O—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of Q are independently replaced by -Cy-, —O—, —NH—, —S—, or —C(O)—, wherein:
-Cy- is an optionally substituted 5-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur;
R^{2a} is a mono-protected amine, a di-protected amine, —NHR³, —N(R³)₂, —NHC(O)R³, —NR³C(O)R³, —NHC(O)NHR³, —NHC(O)N(R³)₂, —NR³C(O)NHR³, —NR³C(O)N(R³)₂, —NHC(O)OR³, —NR³C(O)OR³, —NHSO₂R³, or —NR³SO₂R³; and
each R³ is independently an optionally substituted group selected from aliphatic, a 5-8 membered saturated, partially unsaturated, or aryl ring having 0-4 heteroatoms independently selected from nitrogen, oxygen, or sulfur, an 8-10-membered saturated, partially unsaturated, or aryl bicyclic ring having 0-5 heteroatoms independently selected from nitrogen, oxygen, or sulfur, or a detectable moiety, or:
two R³ on the same nitrogen atom are taken together with said nitrogen atom to form an optionally substituted 4-7 membered saturated, partially unsaturated, or aryl ring having 1-4 heteroatoms independently selected from nitrogen, oxygen, or sulfur.

14. The method according to claim 8, wherein R¹ is a group of formula V: 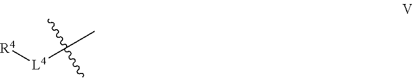
R⁴ is hydrogen, halogen, NO₂, CN, N₃, —N═C═O, —C(R)═NN(R)₂, —P(O)(OR)₂, —P(O)(X″)₂, a 9-30 membered crown ether, a small molecule drug, or an optionally substituted group selected from aliphatic, a 3-8 membered saturated, partially unsaturated, or aryl ring having 0-4 heteroatoms independently selected from nitrogen, oxygen, or sulfur, an 8-10 membered saturated, partially unsaturated, or aryl bicyclic ring having 0-5 heteroatoms independently selected from nitrogen, oxygen, or sulfur, or a detectable moiety;
each X″ is independently halogen;
each R is independently hydrogen or an optionally substituted aliphatic group;
L⁴ is a valence bond or a bivalent, saturated or unsaturated, straight or branched C₁₋₁₂ alkylene chain, wherein 0-6 methylene units of L⁴ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

15. The method according to claim 8 wherein R¹ is selected from any of the groups depicted in Tables 2 and 3.

16. A compound of formula II-e: 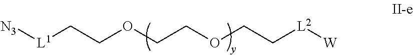
or a salt thereof, wherein:
W is —C(═O)OH, —C(═O)X, —P(═O)(OH)₂, —P(═O)(OR^{a})(OH), —P(═O)(X)₂, —P(═O)(OR^{a})X, —P(═O)(R^{a})OH, —P(═O)(R^{a})X, —O—S(═O)₂OH, —S(═O)₂OH, C(═O)H, —N═C═S, —N═C═O, a phenol, a thiophenol, a Michael acceptor, an epoxide, a suitable leaving group, or an activated ester;
each X is independently Cl, Br, or I; and
each R^{a} is hydrogen, an optionally substituted aliphatic or aryl group, or a a natural or synthetic polymer or copolymer group or a small molecule organic group;
y is 1-2500;
each R is independently hydrogen or an optionally substituted aliphatic group;
L¹ is a valence bond or a bivalent, saturated or unsaturated, straight or branched C₁₋₁₂ alkylene chain, wherein 0-6 methylene units of L¹ are independently replaced by -Cy-, —O—, —NR—, —S—, —OC(O)—, —C(O)O—, —C(O)—, —SO—, —SO₂—, —NRSO₂—, —SO₂NR—, —NRC(O)—, —C(O)NR—, —OC(O)NR—, or —NRC(O)O—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur; and
L² is a valence bond or a bivalent, saturated or unsaturated, straight or branched C₁₋₁₂ alkylene chain, wherein 0-6 methylene units of L² are independently replaced by -Cy-, —O—, —NR—, —S—, or —C(O)—, wherein:
each -Cy- is independently an optionally substituted 3-8 membered bivalent, saturated, partially unsaturated, or aryl ring having 0-4 heteroatoms independently selected from nitrogen, oxygen, or sulfur, or an optionally substituted 8-10 membered bivalent saturated, partially unsaturated, or aryl bicyclic ring having 0-5 heteroatoms independently selected from nitrogen, oxygen, or sulfur.

17. An implantable device, wherein at least a portion of said device is a covalently modified metal surface.

18. The implantable device according to claim 17, wherein said device is PEGylated.

19. The implantable device according to claim 17, wherein said device is selected from cardiovascular devices (e.g., implantable venous catheters, venous ports, tunneled venous catheters, chronic infusion lines or ports, including hepatic artery infusion catheters, pacemaker wires, implantable defibrillators); neurologic/neurosurgical devices (e.g., ventricular peritoneal shunts, ventricular atrial shunts, nerve stimulator devices, dural patches and implants to prevent epidural fibrosis post-laminectomy, devices for continuous subarachnoid infusions); gastrointestinal devices (e.g., chronic indwelling catheters, feeding tubes, portosystemic shunts, shunts for ascites, peritoneal implants for drug delivery, peritoneal dialysis catheters, implantable meshes for hernias, suspensions or solid implants to prevent surgical adhesions, including meshes); genitourinary devices (e.g., uterine implants, including intrauterine devices (IUDs) and devices to prevent endometrial hyperplasia, fallopian tubal implants, including reversible sterilization devices, fallopian tubal stents, artificial sphincters and periurethral implants for incontinence, ureteric stents, chronic indwelling catheters, bladder augmentations, or wraps or splints for vasovasostomy); otolaryngology devices (e.g., ossicular implants, Eustachian tube splints or stents for glue ear or chronic otitis as an alternative to transtempanic drains); and orthopedic implants (e.g., cemented orthopedic prostheses).

20. The implantable device according to claim 19, wherein said device is a stent.

21. A method for expanding the lumen of a body passageway, comprising inserting a stent into the passageway, the stent having a generally tubular structure, at least a portion of the surface of the structure being covalently modified according to the method of claim 1, such that the passageway is expanded.
